(19) United States
(12) Patent Application Publication
(10) Pub. No.: US 2024/0401020 A1
(43) Pub. Date: Dec. 5, 2024

(54) METHODS FOR PREVENTING INTER-DROPLET TRANSFER OF A FLUORESCENT PRODUCT IN AQUEOUS DROPLETS BY FORMING A SOLUBLE COMPLEX WITH CYCLODEXTRIN

(71) Applicant: PATTERN BIOSCIENCE, INC., Austin, TX (US)

(72) Inventor: Ross JOHNSON, Austin, TX (US)

(21) Appl. No.: 18/699,193

(22) PCT Filed: Oct. 6, 2022
(86) PCT No.: PCT/US2022/077648

§ 371 Date: Apr. 5, 2024

Related U.S. Application Data
(60) Provisional application No. 63/252,953, filed on Oct. 6, 2021.

(51) Int. Cl. C12N 11/04; C09K 11/06

(52) U.S. Cl. CPC C12N 11/04; C09K 11/06; C09K 2211/1018

(57) ABSTRACT

A method for retaining fluorescent molecules within aqueous droplets suspended in an emulsion includes combining a plurality of living cells, a plurality of fluorescent molecules, and a plurality of cyclodextrin molecules in an aqueous solution. The method also includes emulsifying the aqueous solution with a hydrophobic fluid to form the aqueous droplets. At least one of the aqueous droplets includes (i) a single living cell or a single cell species in a homogenous aggregate of the plurality of living cells, (ii) at least one fluorescent molecule of the plurality of fluorescent molecules, and (iii) at least one cyclodextrin molecule of the plurality of cyclodextrin molecules.

### FIELD OF THE DISCLOSURE

Methods and products consistent with the present disclosure generally relate to cellular biology, microbiology, and microfluidics. More particularly, methods and products consistent with the disclosure relate to improving the intensity of fluorescence in droplets and decreasing the leakage of florescent molecules between droplets of an array.

### DISCUSSION OF THE FIELD

In various instances, Disease-Causing Cells (DCCs) are found in quantities below the limit-of-detection of conventional analytical techniques. Thus, methods for identifying DCCs and characterizing their response to treatment typically require either multiplication of the target cells and/or target-dependent amplification of the target cells' molecular contents and/or products, depending on the application.

Due to their high sensitivity compared to other techniques, nucleic acid amplification (e.g., PCR-based) tests (NATs) have become the preferred method for fast pathogen identification. DNA can be amplified from a single copy to billions of copies within hours using NATs, such as PCR.

NATs require complicated sample workflow steps which usually include cell lysis followed by a nucleic acid concentration step that also removes PCR inhibitors from the samples. Cell lysis creates an asymmetry in the requirements for nucleic acid extraction efficiency. Mycobacteria and fungi, for example, possess a very thick cell wall compared to gram-negative bacteria and thus are far more difficult to lyse, usually requiring a mechanical lysis step to efficiently disrupt their cell wall. Consequently, NATs that utilize simple chemical lysis methods often lack sensitivity for these tougher pathogens. Furthermore, cell lysis reagents can inhibit PCR reactions because the reagents denature proteins and PCR utilizes proteins to perform the amplification reaction. Therefore, cell lysis introduces the need for highly efficient wash steps to remove lysis reagents from the extracted nucleic acid. In addition, NATs require expensive assay development methods because they rely on pathogen-specific reporter molecules (primers and probes) that must be designed specifically for each target. Each NAT must therefore include an expensive molecular R&D process which involves primer/probe design and screening for each target.

NATs that include two or more targets (multiplexed) cannot simultaneously quantify those targets accurately and precisely. This is because the same nonspecific interactions between reporter species cause variances in the PCR signal output and quantitative PCR relies on reproducible reaction results in order to correlate the generated amplification curves to the initial target concentration. This is a significant limitation because it limits the use of multiplex NATs for the diagnosis of infections from non-sterile infection areas of the body where most infection occur. In non-sterile areas of the body humans are often colonized by the same pathogens that can cause an infection. Because infections are caused by microbes that overwhelm the body's defenses, they will generally produce more colony isolates than commensals will. In non-sterile infection sites, therefore, the number of pathogens present in the clinical sample (the pathogen load) is what determines whether a bacterial species is causing an infection or “peacefully” colonizing the site. For example, in order to definitively diagnose the source of a pneumonia infection from a lower respiratory system (e.g., bronchoalveolar lavage (BAL)) the pathogen load for any bacteria present in the sample must exceed 103 CFU/mL to be considered the source of an infection. Similarly, for urine specimens the threshold is 105 CFU/mL.

Furthermore, if a NAT assay includes more than one target (multiplex assay), requiring more than one reporter species (primer and/or probe pair), the different target and/or reporter species can interact nonspecifically with one another, causing either false positives when the reporter amplifies nonspecifically against the other reagents or false-negatives when the target amplification reaction is inhibited by a non-specific interaction with another species. Consequently, this limits how many targets can be identified within a single NAT. This becomes particularly relevant to the issue of drug resistance because, in the case of gram-negative bacteria and mycobacteria, there are numerous mutations (each mutation being a target) indicative of resistance. NATs can only interrogate a small fraction of those mutations within a single test. In addition, the genes that reside in an organism's genotype may not always contribute to the phenotype. Therefore, genotypic information often portrays an inaccurate or incomplete picture of a pathogen's phenotypic response. Methicillin-resistant Staphylococcus aureus (MRSA), for example, often do not express the mecA gene that confers resistance. Therefore, when it comes to the clinically important determination of whether the pathogen causing an infection is susceptible to a particular drug NATs have low clinical validity because these tests can only examine a small fraction of the genetic mutations that can confer antimicrobial resistance and cannot account for epigenetic resistance mechanisms at all.

NATs must also be engineered to interrogate well understood genetic resistance markers. However, microbes continuously evolve new resistance mechanisms under the selective pressure introduced by antimicrobial drugs. By relying on the information that changes over time (the genotype), NATs become less accurate as microbes evolve new resistance mechanisms. In order to keep up with the fundamentally changing landscape of antimicrobial resistance, new resistance mechanisms must be investigated and understood, and new NATs must be developed and must clear regulatory hurdles to enter the clinic. This costly process can take years.

Phenotypic antimicrobial susceptibility testing (AST) remains the gold standard for diagnosing microbial infections because it measures the phenotypic microbial response to the antimicrobial drugs being considered for treatment, and thus relies on functional endpoints that translate directly to the desired clinical response. Being phenotypic, AST encompasses all resistance mechanisms, thus providing high clinical validity.

AST results in the determination of a minimum inhibitory concentration (MIC), which corresponds to the minimum concentration of an antimicrobial needed to significantly inhibit microbial growth. Because different microbes have different intrinsic characteristics that influence their observed response to a given antimicrobial drug, a MIC alone does not inform the clinician whether pathogens are susceptible or resistant to the antibiotic. For example, an oxacillin MIC of 2 μg/mL indicates susceptibility to oxacillin if the pathogen is Staphylococcus aureus (which would therefore respond to oxacillin treatment) and resistance if the pathogen is Staphylococcus epidermidis (which would therefore not respond to treatment). To interpret any AST result pathogen identification (ID) is required.

AST cannot be accurately carried out if multiple pathogens are present in the same test because microbial growth between species is not distinguishable and the antibiotic response of a species cannot be specifically measured. Furthermore, competition for resources can be enhanced under the selective pressure of an antimicrobial drug, obfuscating the impact of the antimicrobial itself. This is particularly important because most bacterial infections occur in areas of the body that are always populated by bacteria. For infections in non-sterile sites, it is critical to identify and separate the bacteria causing the infection (pathogens) from those that “peacefully” reside at the infection site (commensals). This prevents culturing of the specimen in liquid broth media that are simpler to use and often accelerate microbial growth. Instead, quantitative or semiquantitative culturing is required, which is accomplished by spreading the sample across a petri dish at different concentrations so that at some point on the plate the microbes will grow in isolated colonies of a single species. Plate-streaking also allows lab technician to count visually distinguishable colonies present at a certain concentration to determine whether the microbes in the sample have become invasive. Colonies formed by different species can be distinguished visually from one another and counted. The lab technician can also manually extract a pathogen colony made up of entirely the same species for further testing. Thus, the quantitative/semi-quantitative culturing process not only produces the large number of cells needed for subsequent AST, it also ensures that the bacteria being tested are homogenous and are those causing the infection (pathogens).

However, quantitative/semi-quantitative culturing is highly subjective and error prone, requiring highly skilled technicians to determine which colonies to exclude or include in subsequent testing. Polymicrobial infections are particularly challenging. Often, one of the infecting pathogens is a fastidious organism (an organism that has a complex nutritional requirement and typically only grows under specific conditions) or a slow growing organism and the other is not.

The non-fastidious/faster growing organism will often drown out the fastidious/slower-growing organism on a culture plate and conceal its presence in the specimen.

Because AST requires an upfront culture growth step to produce the large number of cells needed for evaluation against drug candidates for treatment, it can take one to five days of incubation on a petri dish to grow the microbial population from the numbers present in the patient sample to the numbers needed for AST, far too slow to effectively guide antibiotic treatment decision, particularly for serious bacterial infections.

One way to produce evaluation results at significantly faster turn-around-times (<4 to 6 hours (hrs)) than other methods is to make use of microfluidics. The increase in speed is accomplished, in part, by the rapid signal concentration made possible in confined volumes which are orders of magnitude smaller than the milliliter and microliter volumes typically used by other methods.

Individual cells or microbes are isolated or compartmentalized into separate droplets or volumes, enabling quantification to become the simple matter of counting those droplets or volumes associated with a signal indicating the presence of a particular cell or microbe. The shape or change of the signal over time (e.g., a waveform), which is one of the characteristics relied upon for identification and characterization, is orthogonal to the method for quantification-one does not affect the other. Thus, accurate and precise multiplexed identification and quantification is accomplished simultaneously.

Live cells are suspended with cell reagents and/or cell reactants. The suspension is compartmentalized into pico-scale compartments that are observable in real-time. Observed changes for each compartment are recorded into a waveform that can be classified into useful information about the encapsulated cell. The term “cell reagents” includes any substance or mixture of substances used to observe phenotypic variation. Cell reagents act as indicators or detectors of conditions in the compartment without altering or modifying the metabolism or phenotype of a cell. Cell reagents include viability dyes, fluorogenic enzyme substrates, and luminogenic enzyme substrates. The term “cell reactants” includes any substance or mixture of substances used to induce phenotypic variation-essentially any substance that interacts with the cell that is not directly measured during analysis. Cell reactants can influence or modify the metabolism or phenotype of a cell. Cell reactants include cell nutrients, antimicrobial drugs, and cytotoxic drugs. Cell nutrients include, but are not limited to culture media, buffers, salts, gases (e.g., O2 and CO2), hormones, or the like. Cell nutrients also include any carbohydrates, sugars, and alcohols such as ribitol/adonitol, glucose, maltose, mannose, palatinose, sucrose, galactose, ribose, raffinose, trehalose, gentiobiose, lactose, melibiose, rhamnose, sorbose, turanose, xylose, and/or melizitose. Cell reactants can also include enzyme inhibitors and the like. Drugs include, but are not limited to small molecules, antimicrobial drugs, chemotherapeutic drugs, cytotoxic drugs, bacteriophages, peptides, nanoparticles, CRISPR-CAS 9-based therapeutics, immunotherapies, and the like.

However, in the context of microfluidics, there can be a tendency for the markers such as viability dyes, fluorogenic enzyme substrates, or luminogenic enzyme substrates to bleed between neighboring droplets. This can lower the accuracy of the evaluation results produced.

Some additives such as bovine serum albumin (BSA), dextran, or sucrose have been used to reduce migration of fluorescent markers between droplets. However, additives such as BSA, dextran, and sucrose at best provide only minimal improvements in reducing dye leakage. Additionally, these additives have a tendency to slow the signal generation rate.

Thus, there remains a need for additional methods to prevent fluorescent markers from migrating between droplets in microfluidic arrays and that do so without interfering with the reaction that occurs in the presence of a live cell to convert the fluorescent molecules from non-fluorescent to fluorescent, or vice versa.

### SUMMARY

The disclosure provides a method for retaining fluorescent molecules within aqueous droplets suspended in an emulsion comprising: combining a plurality of living cells, a plurality of fluorescent molecules, and a plurality of cyclodextrin molecules in an aqueous solution; and emulsifying the aqueous solution with a hydrophobic fluid to form the aqueous droplets; wherein at least one of the aqueous droplets includes (i) a single living cell or a single cell species in a homogenous aggregate of the plurality of living cells, (ii) at least one fluorescent molecule of the plurality of fluorescent molecules, and (iii) at least one cyclodextrin molecule of the plurality of cyclodextrin molecules.

In some embodiments, the plurality of living cells includes at least one of bacterial cells, fungal cells, plant cells, and animal cells.

In some embodiments, the plurality of living cells includes living cells of at least one of Klebsiella pneumoniae, Escherichia coli, and Serratia marcescens.

In some embodiments, the plurality of living cells includes human cells.

In some embodiments, the human cells are derived from sputum, saliva, urine, blood, cerebrospinal fluid, seminal fluid, stool, or tissue.

In some embodiments, the hydrophobic fluid comprises an oil.

In some embodiments, the oil is a fluorocarbon oil.

In some embodiments, fluorescent molecules are hydrophobic fluorescent molecules.

In some embodiments, the hydrophobic fluorescent molecules are resazurin dye molecules, acridine molecules, tetrazolium dye molecules, coumarin dye molecules, anthraquinone dye molecules, cyanine dye molecules, azo dye molecules, xanthene dye molecules, arylmethine dye molecules, pyrene derivative dye molecules, or ruthenium bipyridyl complex dye molecules.

In some embodiments, the cyclodextrin molecules are α-cyclodextrin molecules, β-cyclodextrin molecules, or γ-cyclodextrin molecules.

In some embodiments, the fluorescent molecules and the cyclodextrin molecules do not impede viability (i.e., an ability to survive) or metabolic activity (i.e., cellular functions) of the living cells.

In some embodiments, the combining and the emulsifying are performed at a temperature of no greater than 40° C.

In some embodiments, the method also comprises combining a surfactant in the aqueous solution.

In some embodiments, the surfactant is a fluoro-surfactant.

The disclosure also provides an emulsion comprising a plurality of aqueous droplets suspended in a hydrophobic fluid, wherein at least one of the aqueous droplets includes: a single living cell or a single cell species in a homogenous aggregate; at least one fluorescent molecule; and at least one cyclodextrin molecule.

In some embodiments, the single living cell or the single cell species in a homogenous aggregate includes one of bacterial cells, fungal cells, plant cells, and animal cells.

In some embodiments, the single living cell or the single cell species in a homogeneous aggregate includes one of Klebsiella pneumoniae, Escherichia coli, and Serratia marcescens.

In some embodiments, the single living cell or the single cell species in a homogeneous aggregate includes human cells.

In some embodiments, the human cells are derived from sputum, saliva, urine, blood, cerebrospinal fluid, seminal fluid, stool, or tissue.

In some embodiments, the hydrophobic fluid comprises an oil.

In some embodiments, the oil is a fluorocarbon oil.

In some embodiments, the fluorescent molecules are hydrophobic fluorescent molecules.

In some embodiments, the hydrophobic fluorescent molecules are resazurin dye molecules, acridine molecules, tetrazolium dye molecules, coumarin dye molecules, anthraquinone dye molecules, cyanine dye molecules, azo dye molecules, xanthene dye molecules, arylmethine dye molecules, pyrene derivative dye molecules, or ruthenium bipyridyl complex dye molecules.

In some embodiments, the at least one cyclodextrin molecule is at least one α-cyclodextrin molecule, at least one β-cyclodextrin molecule, or at least one γ-cyclodextrin molecule.

In some embodiments, the fluorescent molecules and the cyclodextrin molecules are (i) biocompatible with the single living cell or the single cell species in a homogeneous aggregate, and (ii) do not impede metabolic activity of the a single living cell or the single cell species in a homogeneous aggregate.

In some embodiments, the aqueous droplets further includes a surfactant.

In some embodiments, wherein the surfactant is a fluoro-surfactant.

As used herein the term “sample partition” or “partition” refers to a portion of a sample. A sample can be partitioned into a number of partitions, or sub-sample volumes. Each partition can bet processed, treated, manipulated, and/or incubated separately and under distinct or similar conditions.

As used herein, the term “compartment” refers to a volume of fluid (e.g., liquid or gas) that is a separated portion of a bulk volume (e.g., a sample). A bulk volume may be compartmentalized into any suitable number of smaller volumes or compartments. Compartments may be separated by a physical barrier or by physical forces (e.g., surface tension, hydrophobic repulsion, etc.). Compartments generated from the larger volume may be substantially uniform in size (monodisperse) or may have non-uniform sizes (polydisperse). Compartments may be produced by any suitable manner, including emulsion, microfluidics, and micro spray methods. One example of compartments are droplets.

As used herein, the term “droplet” refers to a small volume of liquid which is immiscible with its surroundings (e.g., gases, liquids, surfaces, etc.). A droplet may reside upon a surface or be encapsulated by a fluid with which it is immiscible, such as the continuous phase of an emulsion, a gas, or a combination thereof. A droplet is typically spherical or substantially spherical in shape but may be non-spherical. The shape of an otherwise spherical or substantially spherical droplet may be altered by deposition onto a surface or by passage through or collection in a chamber having dimensions smaller than the diameter of the droplet. A droplet may be a “simple droplet” or a “compound droplet,” wherein one droplet encapsulates one or more additional smaller droplets. The volume of a droplet and/or the average volume of a set of droplets provided herein is typically less than about one microliter, for example droplet volume can be about 1 μL, 100 nL, 10 nL, 1 nL, 100 fL, 10 fL, 1 fL, including all values and ranges there between. The diameter of a droplet and/or the average diameter of a set of droplets provided herein is typically less than about one millimeter, for example 1 mm, 100 μm, 10 μm, to 1 μm, including all values and ranges there between. Droplets may be formed by any suitable technique, including emulsification, microfluidics, etc., and may be monodisperse, substantially monodisperse (differing by less than 5% in diameter or volume), or polydisperse.

Other embodiments are discussed throughout this application. Any embodiment discussed with respect to one aspect applies to other aspects as well and vice versa. Each embodiment described herein is understood to be embodiments that are applicable to all aspects. It is contemplated that any embodiment discussed herein can be implemented with respect to any method or composition of the disclosure, and vice versa. Furthermore, compositions and kits can be used to achieve methods of the disclosure.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or if the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”), and “containing” (and any form of containing, such as “contains” and “contain”) are inclusive or open-ended and do not exclude additional, unrecited elements or method steps.

In the following discussion and in the claims, the terms “including” and “comprising” are used in an open-ended fashion, and thus should be interpreted to mean “including, but not limited to.” Also, the terms “coupled,” “link,” “points to,” and forms thereof, are intended to mean either an indirect or direct connection. Thus, if a first component links or couples to a second component, that connection may be through a direct connection or through an indirect connection via other components and connections.

Other objects, features and advantages will become apparent from the following detailed description. It should be understood, however, that the detailed description and the specific examples, while indicating specific embodiments, are given by way of illustration only, since various changes and modifications within the spirit and scope of the disclosure will become apparent to those skilled in the art from this detailed description.

### BRIEF DESCRIPTION OF THE DRAWINGS

The accompanying drawings, which are incorporated in and constitute a part of this disclosure, illustrate various embodiments and aspects of the present disclosure. In the drawings:

FIG. 1 illustrates the general concept for rapid phenotypic diagnosis and screening described. Individual cells are compartmentalized so that the observable changes in the compartment represent the functional characteristics of the encapsulated cell. Functional variation is recognized and used to classify the encapsulated cell.

FIG. 2 illustrates the incorporation of the general concept of FIG. 1 into the methods described. Live cells are suspended with cell reagents and cell reactants. The suspension is partitioned into pico-scale compartments that are observable in real-time. Observed changes for each compartment are recorded into a waveform that can be classified into useful information about the encapsulated cell.

FIG. 3 schematically illustrates separating a cell suspension or sample into multiple compartments.

FIG. 4 (left) illustrates an observable compartment signal that is sampled and recorded over time. Empty compartments will produce repeatable background signals over time. Compartments with cells in them will generate real-time changes that are different than those generated from empty droplets or from compartments with different cell phenotypes; (right) The recorded signal waveforms are fed into a classification function, in this case a neural network, that classifies each compartmentalized cell based on the sampled waveform.

FIG. 5 illustrates the basic embodiment of sampling and recording of a phenotypic waveform from an individual compartment within a partition or sub-sample and feeding the recorded waveform into a deep neural network for classification. The bottom is a schematic illustration of the basic embodiment described above.

FIG. 6A-6C (6A) is a schematic illustration of one embodiment that utilizes multiple partitions or sub-samples, each with its own reactant mixture to increase phenotypic variation for a given sample or to account for the nutrient needs of different cell types. This embodiment can be reagent-free as described in more detail below; (6B) is a schematic illustration of a similar embodiment to (6A) but with cell reagents included; (6C) is a schematic illustration of an embodiment with partitions that share the same cell reactants but include different cell reagents.

FIG. 7A-7C are schematics illustrating methods that include a combination of cell reactants and cell reagents across one or more partitions.

FIG. 8A-8B (8A) schematically illustrates an embodiment of FIG. 6B where the cell reactants are different nutrient mixes and a single cell reagent is used; (8B) schematically illustrates an embodiment of FIG. 7B where the cell reactant is a nutrient mix and the cell reagents are different combination of viability dyes, fluorogenic substrates, and luminogenic substrates.

FIG. 9 is a schematic of a diagnostic test for evaluating the impact of a cell reactant on a cell species. The Test Reactant (TR) is only present in the Test Sample (TS) which is otherwise identical to the Control Sample (CS) partition. N1 is an encoder that compresses the full waveforms down into a sparse representation which is then classified by CL. The Characterizer (CH) contains global statistics of both populations such as the average intensity of each compartment, for example. N2 uses the classified waveforms in CL and the population statistics in CH for both TS and CS to compute a clinical endpoint.

FIG. 10 is a schematic of an embodiment where the Test Reactant in FIG. 9 is a drug. Here N2 utilizes the classified waveforms in CL and the population statistics in CH to compute a clinical endpoint associated with prescribing drug that interacts with the cells being tested. Using machine learning, the neural network N2 can be trained to any clinical endpoint.

FIG. 11 shows that both the control and test partition or sub-sample waveforms can be fed into the same N1, CH, and CL, but the resulting statistics are fed into separate input nodes of the Predictor neural network, allowing for a detailed comparison of the variation present in CS and TS.

FIG. 12 is a schematic of an example of the embodiment described in FIG. 10 for multiple drugs.

FIG. 13 illustrates a method for diagnosis using machine learning using sample partition statistics combined with other laboratory results, patient information, and community information to arrive at the most accurate and comprehensive diagnosis and prognosis.

FIG. 14 is an illustration of a droplet monolayer being imaged by a camera and an overhead view of a droplet monolayer. The droplet monolayer provides for good thermal conductivity and temperature control.

FIG. 15 illustrates using shear stress for droplet generation or using Laplace pressure gradients for droplet generation.

FIG. 16 shows the visualization of an isolated bacterium in a droplet monolayer.

FIG. 17 illustrates distinct waveform characteristics between S. aureus and K. pneumoniae with multiple waveforms from a droplet array. The waveforms are clearly distinguishable.

FIG. 18 illustrates waveforms representing various bacterial species.

FIG. 19 shows time lapse images of S. aureus from 0 to 2 hours with and without antibiotic.

FIG. 20 illustrates an example of results from pathogen identification studies.

FIG. 21 is a 40× microscope image of an 80 μm droplet that initially contained a single Klebsiella pneumoniae bacterium. After more than 12 hrs of incubation at 37° C. the bacterial growth in the droplet almost entirely blocks light transmission through the droplet.

FIG. 22 is a flow chart describing a method for retaining fluorescent molecules within aqueous droplets suspended in an emulsion.

FIG. 23A is a graph showing droplet fluorescence intensity with and without the addition of cyclodextrin in the presence of Klebsiella pneumoniae.

FIG. 23B is a microscope image of a droplet array using Klebsiella pneumoniae without the addition of cyclodextrin.

FIG. 23C is a microscope image of a droplet array using Klebsiella pneumoniae with the addition of cyclodextrin.

FIG. 24A is a graph showing droplet fluorescence intensity with and without the addition of cyclodextrin in the presence of Escherichia coli.

FIG. 24B is a microscope image of a droplet array using Escherichia coli without the addition of cyclodextrin.

FIG. 24C is a microscope image of a droplet array using Escherichia coli with the addition of cyclodextrin.

FIG. 25A is a graph showing droplet fluorescence intensity with and without the addition of cyclodextrin in the presence of a first strain of Serratia marcescens.

FIG. 25B is a microscope image of a droplet array using a first strain of Serratia marcescens without the addition of cyclodextrin.

FIG. 25C is a microscope image of a droplet array using a first strain of Serratia marcescens with the addition of cyclodextrin.

FIG. 26A is a graph showing droplet fluorescence intensity with and without the addition of cyclodextrin in the presence of a second strain of Serratia marcescens.

FIG. 26B is a microscope image of a droplet array using a second strain of Serratia marcescens without the addition of cyclodextrin.

FIG. 26C is a microscope image of a droplet array using a second strain of Serratia marcescens with the addition of cyclodextrin.

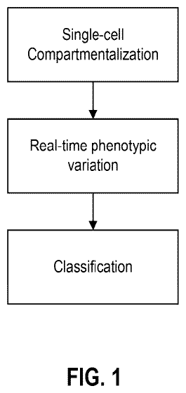

### DETAILED DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to intimate that the scope of the disclosure, including the claims, is limited to that embodiment.

Certain aspects of the disclosure generally relate to methods for sample characterization using single-cell analysis. Sample characterization can be used to diagnose a condition or disease state or to assess and characterize cell phenotypes in the sample, for instance drug susceptibility or resistance. The following sections discuss general considerations for methods of producing and analyzing cellular phenotypes by using compartmentalization of a sample or its sub-samples (i.e., partitions). In certain aspects reagent-free, optical, luminescent, fluorogenic, luminogenic, or other signals can be analyzed and characterized using neural networks and other methods of data analysis.

Analytical methods include the use of artificial intelligence (AI). AI can be used to analyze and/or compare one or more samples to determine one or more cellular phenotypes in a sample. In certain aspects the cellular phenotype(s) can be used in identifying, diagnosing, prognosing, or characterizing a subject or a sample from a subject. The methods can be modified for any clinical endpoint, such as patient outcomes, minimum inhibitory concentration, susceptible or resistant, prognoses such as length of hospital stay, patient risk, etc.

The general concept for rapid phenotypic diagnosis and/or screening includes compartmentalizing individual cells or cellular aggregates so that the observable changes in the compartment represent the functional characteristics of the compartmentalized or encapsulated cell. Variation in cellular function is assessed and/or characterized, and this functional variation or phenotype is used to classify the cell.

Single-cell compartmentalization. Due to the limited-diffusion environment, compartmentalization, or encapsulation within a pico-scale (sub-nanoliter) compartment drastically enriches the concentration of cell components and products within the reaction volume, allowing cell outputs to achieve detectable concentrations much faster than within typical reaction volumes. A single cell in a 500 pico-liter reaction volume, for example, is equivalent to 2 million cells in a milliliter reaction volume. Consequently, compartmentalization allows cell outputs to be observed much sooner and more sensitively than in traditional volumes.

Similarly, the encapsulated or compartmentalized cell can rapidly influence the environmental conditions within the pico-scale compartment. Changes in the compartment's pH or redox potential, for example, will be governed by the encapsulated cell or daughter cells (the result of one or more cell divisions).

Some single-celled microorganisms cannot always be isolated as individual cells, notably bacteria such as Staphylococcus aureus which often present in monoclonal cell aggregates or clusters after cell division. Cell clustering is, in fact, a phenotypic trait that can be used to distinguish clustering organisms from those that do not cluster. Furthermore, the aggregated cells need not be distinguished therapeutically because they will generally respond to the same therapies, drugs, or conditions.

Real-time phenotypic variation. At pico-scale volumes, observable changes in the compartment are dominated by the cell phenotype, including the metabolome, transcriptome, proteome, or environmental variation, examples include:

Metabolome variation. The metabolome comprises the complete set of molecules in the compartment that undergo a chemical transformation in the presence of a live cell, including cell reagent(s) that are compartmentalized along with the cell, including enzyme substrates.

Enzyme substrates comprise any substance catalyzed or modified by an enzyme in the compartment's proteome or transcriptome (protein or RNA). Substrate variation is distinguished here from the proteomic and transcriptomic variation because it is inherently more multi-dimensional.

Substrate reaction rates depend on solution conditions, substrate concentration, and the substrate's access to the enzyme's active site. For example, an enzyme substrate may need to permeate the cell wall and/or membrane to access an enzyme within a cell. Because cell permeability can vary between species, the substrate concentration varies characteristically over time in two compartments encapsulating different cell species even if the proteome and the environment are identical in both compartments.

Finally, while metabolomic variation often includes substrate variation, it should be clear that substrate variation includes enzymes and reagents that do not participate in the cell's metabolic pathways, including any reagent introduced into the compartment that undergoes an enzymatically catalyzed chemical transformation.

Transcriptome variation. The transcriptome comprises the complete set of RNA transcripts in the compartment, including messenger RNA (mRNA) and micro-RNA (miRNA). The concentration of an RNA transcript will vary according to the cell's phenotype and can be used to classify the compartmentalized cell. Micro-RNA is often secreted and can be probed from outside the cell, concentrating quickly in the pico-scale environment and obviating the need for cell permeable detection reagents.

Proteome variation. The proteome comprises the complete set of proteins in the compartment. The compartment's proteomic composition will vary in the presence of a live cell according to the cell phenotype. The proteome can include proteins expressed from foreign or heterologous genes introduced into the cell through transformation, transfection, and transduction.

Environmental variation. Environmental variation comprises any change to the compartment's temperature, pH, and redox potential. At pico-scale volumes, environmental variation is governed by the interaction between the cell and the cell reactants that are compartmentalized along with the cell.

Classification. Single-cell compartmentalization ensures that the phenotypic changes observed in a compartment are characteristic of a single cell species rather than an average of multiple species. These changes can then be classified into useful information using statistical data analysis. The method of the disclosure is compatible with statistical approaches that rely on both supervised learning and unsupervised learning.

Phenotypic diagnosis based on supervised learning. Classification is the problem of identifying to which of a set of categories a phenotypic observation belongs based on a training set of observations whose category membership is known. Classification is considered an instance of supervised learning where a training set of correctly identified observations is available. An example is assigning a diagnosis to a given patient based on the observed characteristics of a patient sample using a function based on previous observations under relevant conditions. The methods of the disclosure are compatible with any statistical classification method based on supervised learning, including neural networks, linear vector quantization, linear classifiers, support vector machines, quadratic classifiers, decision trees, kernel estimation, and meta-algorithm approaches. Certain aspects use one or more neural networks.

Phenotypic diagnosis based on unsupervised learning. Statistical analyses based on unsupervised learning are useful in instances when relative categorization possesses predictive value. For example, if multiple drugs are tested on a single cell species, the relative impact of each drug may predict the best drug candidate(s).

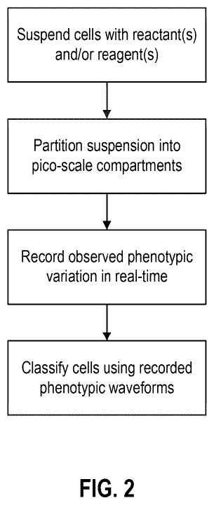

Rapid Phenotype Assessment. FIG. 2 diagrams a method for rapid phenotypic diagnosis. Live cells are suspended with cell reagents and/or cell reactants. The suspension is compartmentalized into pico-scale compartments that are observable in real-time. Observed changes for each compartment are recorded into a waveform that can be classified into useful information about the encapsulated cell. The term “cell reagents” includes any substance or mixture of substances used to observe phenotypic variation. Cell reagents act as indicators or detectors of conditions in the compartment without altering or modifying the metabolism or phenotype of a cell. Cell reagents include viability dyes, fluorogenic enzyme substrates, and luminogenic enzyme substrates. The term “cell reactants” includes any substance or mixture of substances used to induce phenotypic variation-essentially any substance that interacts with the cell that is not directly measured during analysis. Cell reactants can influence or modify the metabolism or phenotype of a cell. Cell reactants include cell nutrients, antimicrobial drugs, and cytotoxic drugs. Cell nutrients include, but are not limited to culture media, buffers, salts, gases (e.g., O2 and CO2), hormones, or the like. Cell nutrients also include any carbohydrates, sugars, and alcohols such as ribitol/adonitol, glucose, maltose, mannose, palatinose, sucrose, galactose, ribose, raffinose, trehalose, gentiobiose, lactose, melibiose, rhamnose, sorbose, turanose, xylose, and/or melizitose. Cell reactants can also include enzyme inhibitors and the like. Drugs include, but are not limited to small molecules, antimicrobial drugs, chemotherapeutic drugs, cytotoxic drugs, bacteriophages, peptides, nanoparticles, CRISPR-CAS 9-based therapeutics, immunotherapies, and the like.

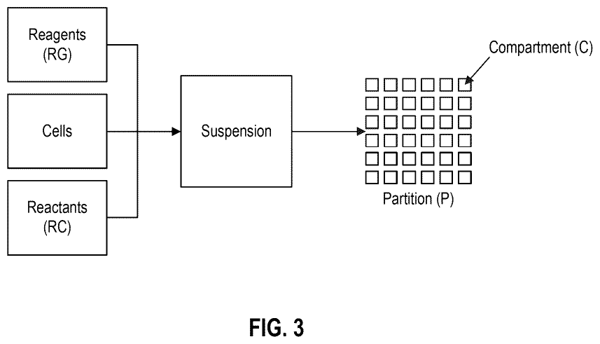

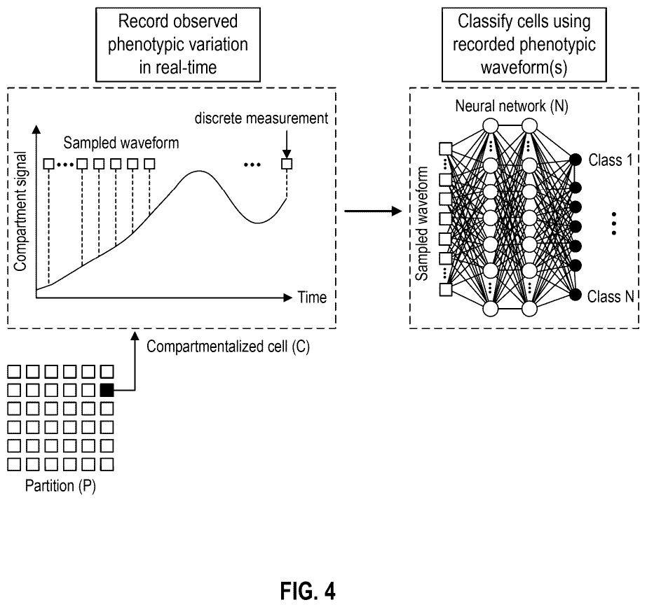

In various aspects a sample is partitioned or compartmentalized (FIG. 3). A sample or cell suspension can be processed into multiple compartments. Each compartment is then characterized (FIG. 4). On the left side of FIG. 4, an observable compartment signal is sampled and recorded over time. Empty compartments will produce repeatable background signals over time. Compartments with cells in them will generate real-time changes that are different than those generated from empty droplets or from compartments with different cell phenotypes. The recorded signal waveforms are fed into a classification function, in this case a neural network, that classifies each compartmentalized cell based on the sampled waveform.

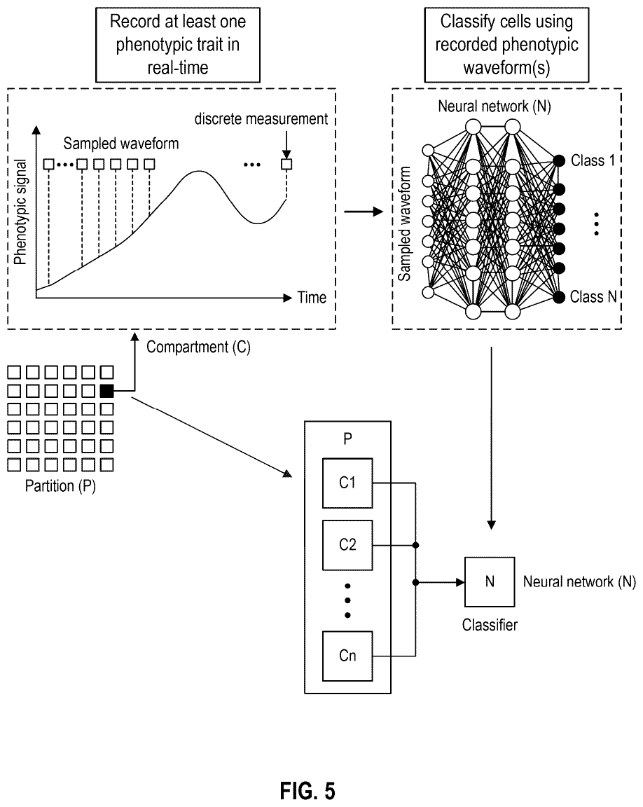

FIG. 5 illustrates a basic embodiment of methods described herein. FIG. 5 illustrates one example of the sampling and recording of a phenotypic waveform from an individual compartment within a partition (sub-sample or portion of the sample) and feeding the recorded waveform into a deep neural network for classification.

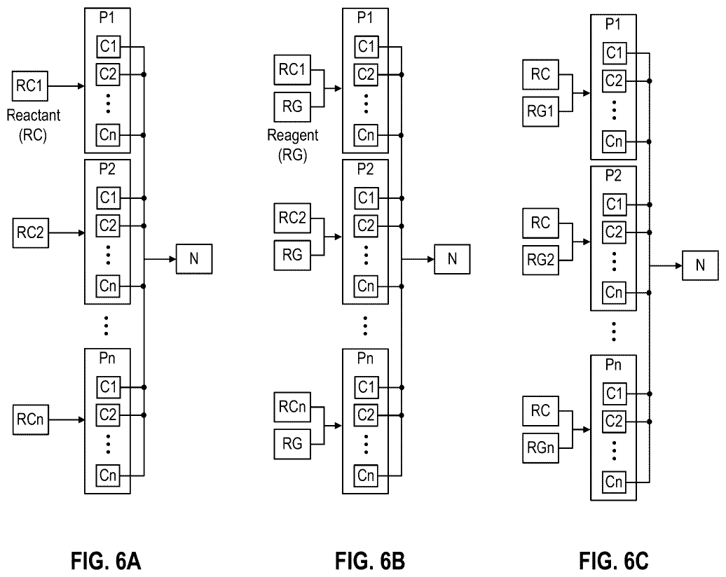

FIG. 6 illustrates various schemes that can be performed using one or more reagents and/or one or more reactants. FIG. 6A illustrates a method that utilizes multiple partitions, each with its own reactant mixture to increase phenotypic variation for a given sample or to account for the nutrient needs of different cell types. In particular embodiments the scheme is reagent-free. FIG. 6B is a schematic illustration of a similar embodiment to FIG. 6A used in conjunction with cell reagents. FIG. 6C is a schematic illustration of a scheme with partitions that share the same cell reactants but include different cell reagents. Methods capable of reagent free detection include:

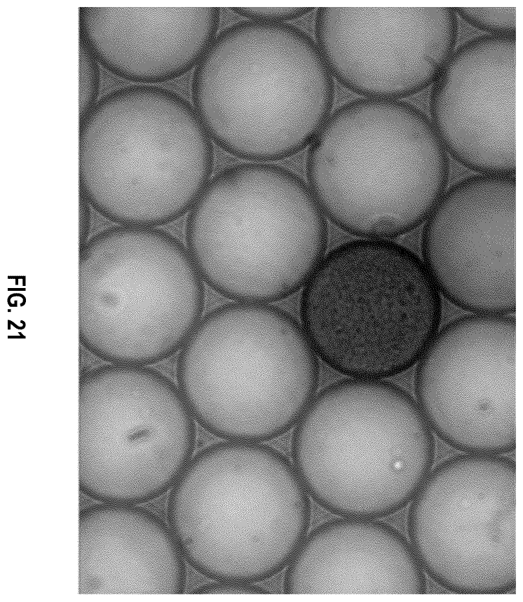

Optical density of the compartment. As cells grow, they will impact the optical density (OD) of the compartment, which will vary depending on growth characteristics of the cell and those growth characteristics as determined by OD could be used to classify the cell (see for example FIG. 21). OD method is particularly well suited for a two-dimensional droplet array using simple brightfield microscopy.

Spectral absorbance of the compartment. There are many ways to accomplish spectroscopy. In a basic approach, a sample is exposed to a white beam source and a detector is used to establish a baseline and then the emitter/detector assembly scans across a two-dimensional array of droplets and an absorption spectrum is obtained by comparing the transmitted spectra to the baseline.

Redox potential or pH. Compartmentalization methods that involve etching compartments into silicon can incorporate circuitry to measure the changing redox potential and pH of the compartment.

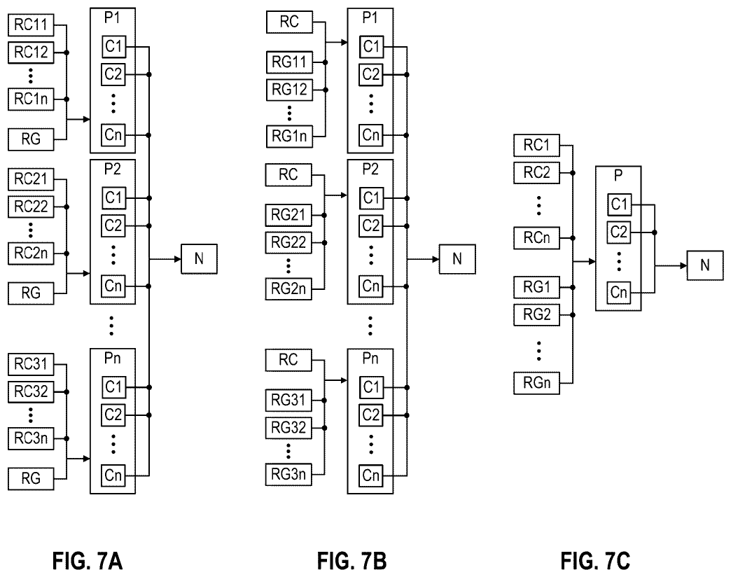

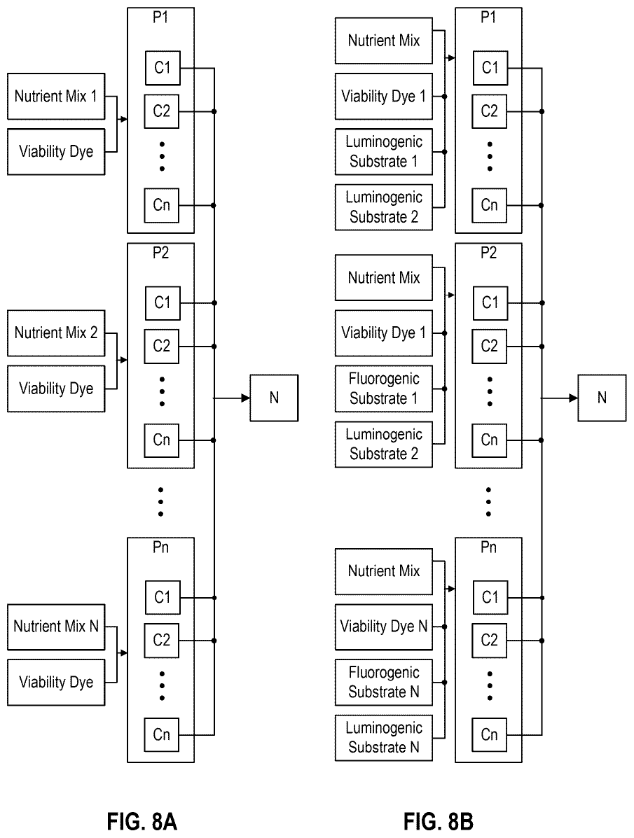

Certain schemes can use multi-reagent and multi-reactant partitions. FIG. 7A to FIG. 7C are schematics illustrating schemes that include a combination of cell reactants and cell reagents across one or more partitions. One particular scheme is illustrated in FIG. 8. FIG. 8A illustrates one embodiment of the scheme illustrated in FIG. 6B where the cell reactants are different nutrient mixes and a single cell reagent is used. FIG. 8B illustrates one embodiment of the scheme illustrated in FIG. 7B where the cell reactant is a nutrient mix and the cell reagents are different combinations of viability dyes, fluorogenic substrates, and luminogenic substrates.

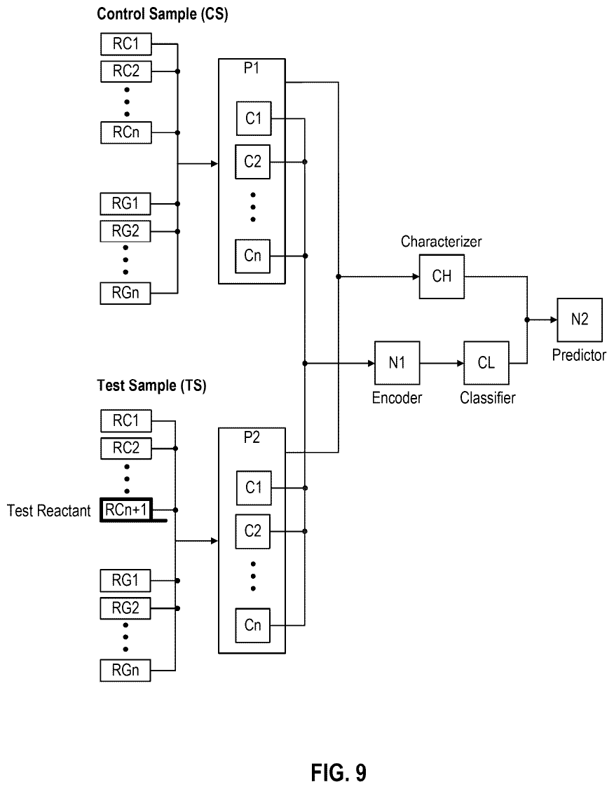

In certain aspects the methods described herein can be used for rapid phenotypic testing of a cell reactant, such as evaluating a test substance for an effect on a cell. FIG. 9 is a schematic of a diagnostic test for evaluating the impact of a cell reactant on a cell species. The Test Reactant (TR) is only present in the Test Sample (TS) which is otherwise identical to the Control Sample (CS) partition. N1 is an encoder that compresses the full waveforms down into a sparse representation which is then classified by CL. The Characterizer (CH) contains global statistics of both populations such as the average intensity of each compartment, for example. N2 uses the classified waveforms in CL and the population statistics in CH for both TS and CS to compute a clinical endpoint.

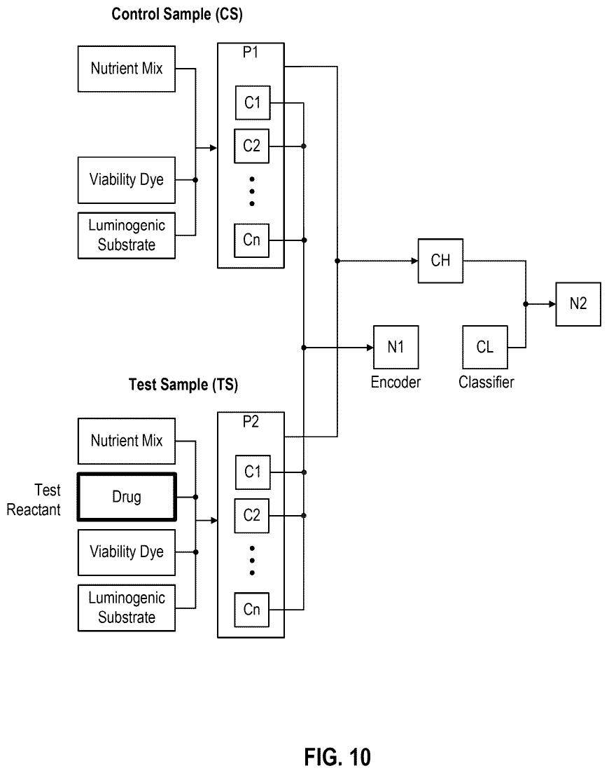

FIG. 10 is a schematic of scheme where the Test Reactant in FIG. 9 is a drug. Here N2 utilizes the classified waveforms in CL and the population statistics in CH to compute a clinical endpoint associated with prescribing a drug that interacts with the cells being tested. Using machine learning, the neural network N2 can be trained to any clinical endpoint. Examples include patient diagnosis, patient prognosis, and the like.

Patient Diagnosis. One advantage of the methods described herein is that it allows a diagnostic test to be directly modified for any clinical endpoint that is relevant to the cells being tested.

Minimum Inhibitory Concentration (MIC). In microbiology, the minimum inhibitory concentration (MIC) is the lowest concentration of a chemical that prevents visible growth of a microbe. MIC information can be used to calculate an optimal dose for a given antimicrobial, depending on the site of infection.

Susceptible, Intermediate, or Resistant (SIR). In microbiology, a MIC result is interpreted as susceptible (or sensitive), intermediate, or resistant for a given pathogen-antimicrobial combination by using cutoff points specific to that combination. MIC cutoffs are established by CLSI guidelines for each pathogen class. Traditionally, an antimicrobial susceptibility test (AST) produces a MIC for a given antimicrobial drug and a separate test is used to identify (ID) the pathogen being tested. The pathogen ID is used to establish the SIR cutoffs for the antimicrobial drug being tested. Only after the ID is known can the cutoffs be established to understand whether the MIC indicates resistance or susceptibility.

An advantage to the methods described herein is that the information in the CS sample can be used to simultaneously establish a MIC and SIR cutoffs without requiring an intermediate ID step that translates the waveforms in the CS into a taxonomic classification. This enables the entire system to be directed to the clinical endpoint (SIR) rather than two intermediary steps.

Pharmacodynamics. Antimicrobial MICs are normally a way of predicting pharmacodynamics of the drug. The MIC is a measure of the potency of an antimicrobial drug. Isolates of a particular species will have varying MICs; sensitive strains will have relatively low MICs, and resistant strains will have relatively high MICs. The breakpoint MIC is the MIC that separates sensitive and resistant strains, and it was traditionally selected based on its ability to distinguish two disparate populations: one population with MICs at less than the breakpoint (i.e., susceptible) and one with MICs at more than the breakpoint (i.e., resistant).

Another attribute of the breakpoint MIC is correspondence to achievable serum drug levels using standard dosing. To guide dosing strategy MIC can be combined with intrinsic antibiotic information such as whether the antimicrobial drug is time-dependent or concentration dependent.

Patient Antibiogram. An antibiogram is an SIR result for each antibiotic in a set of antimicrobials being considered for treatment, thus providing an overall profile of antibiotic susceptibility testing results of a specific microorganism to a battery of drugs. A plurality of drugs can be used and each profile for each drug documented.

Chemosensitivity Testing and Mammalian Cell Diagnosis. The methods described above for antimicrobial susceptibility testing are directly applicable to chemosensitivity or drug sensitivity for cancer as well as for tissue-based diseases where the tissue/cells can be isolated and exposure or contact with a drug alters cellular characteristics.

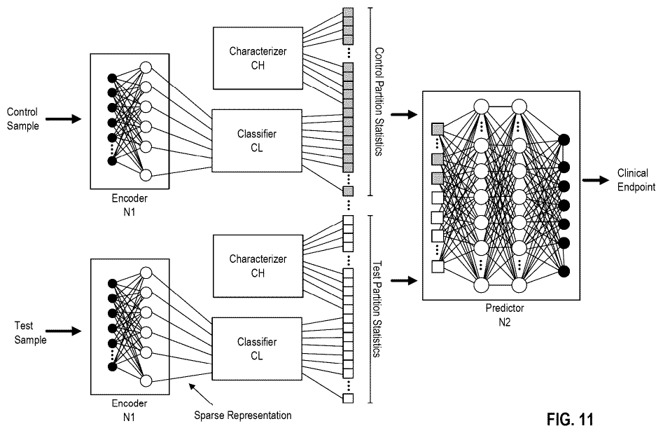

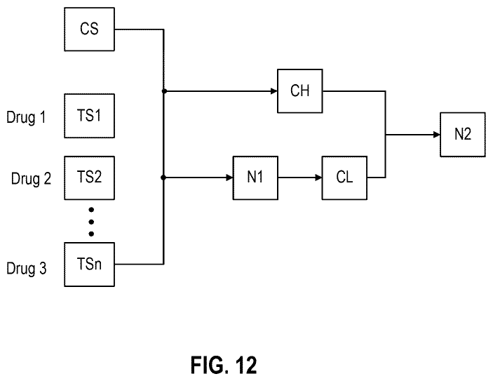

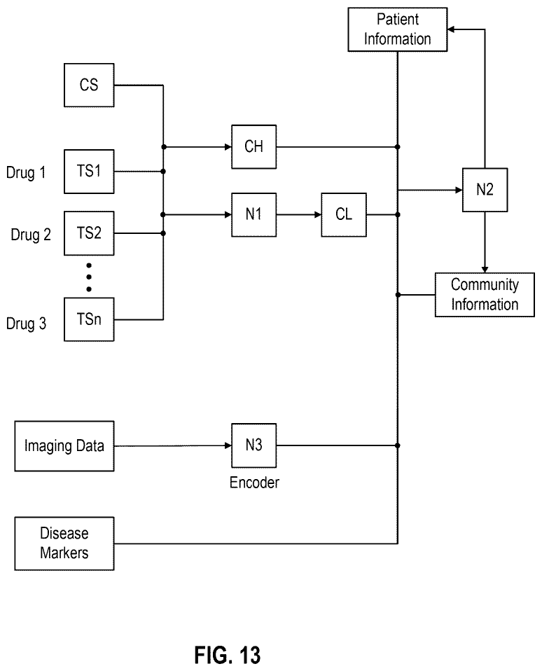

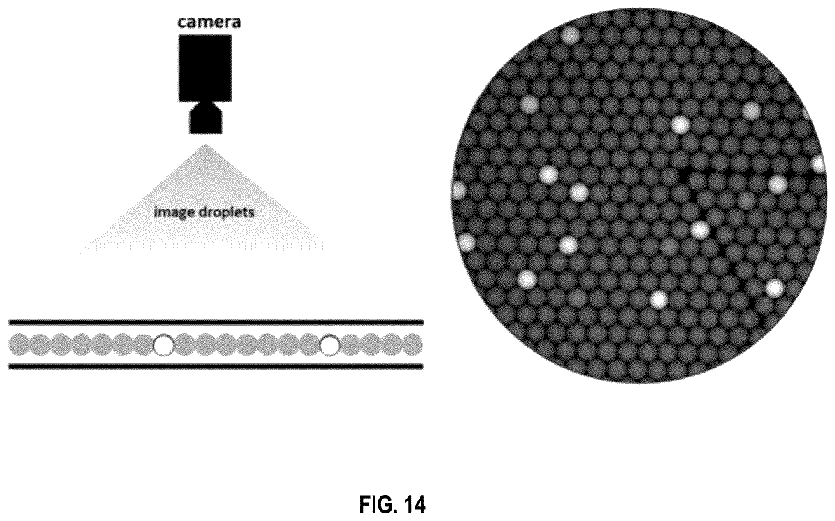

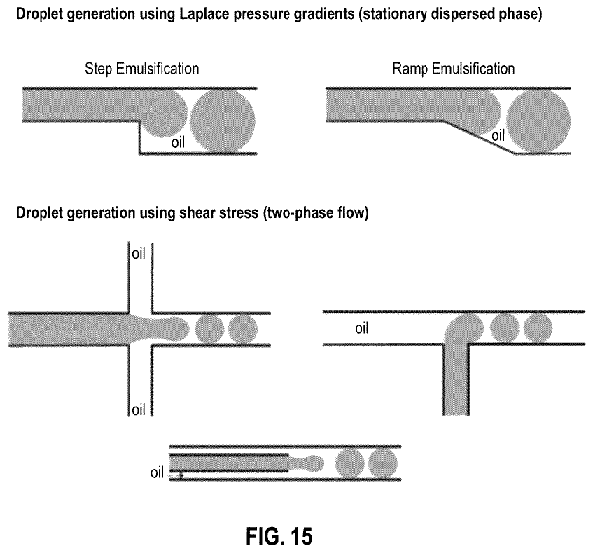

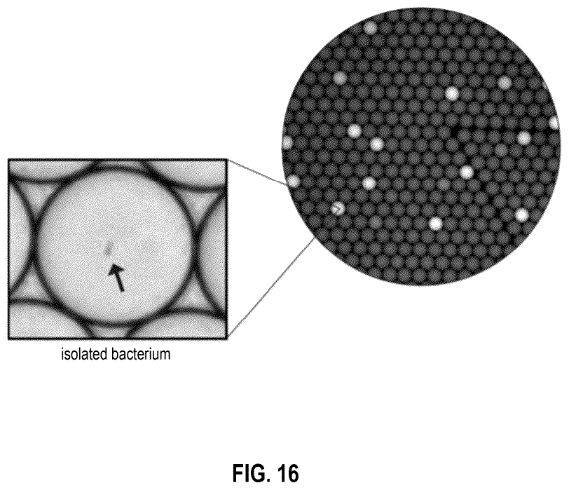

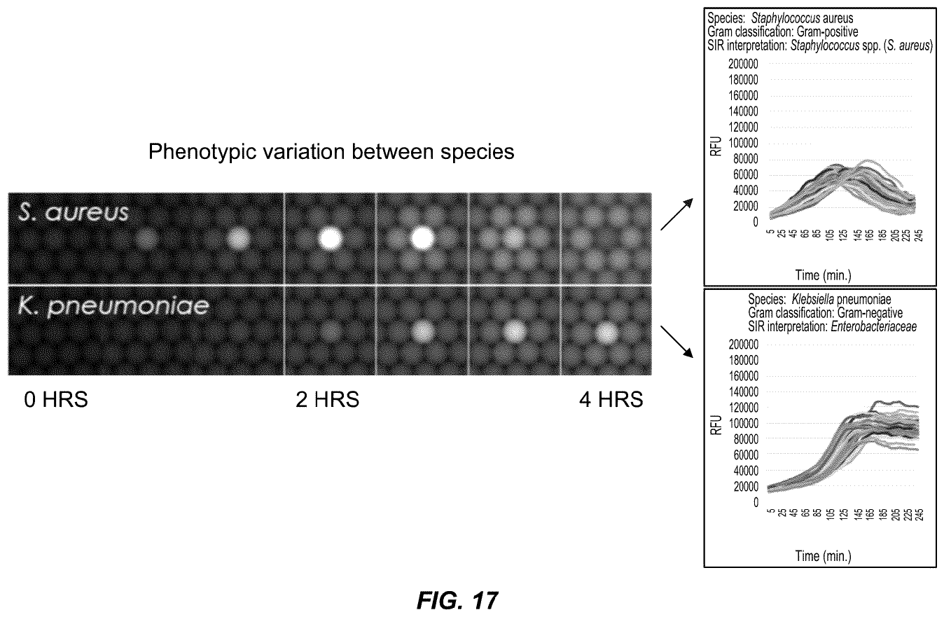

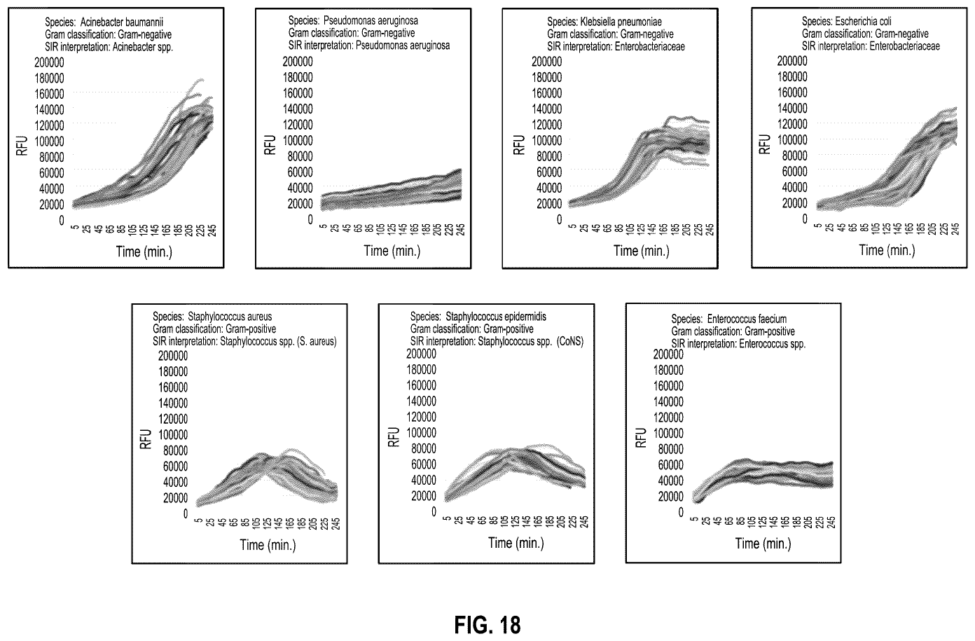

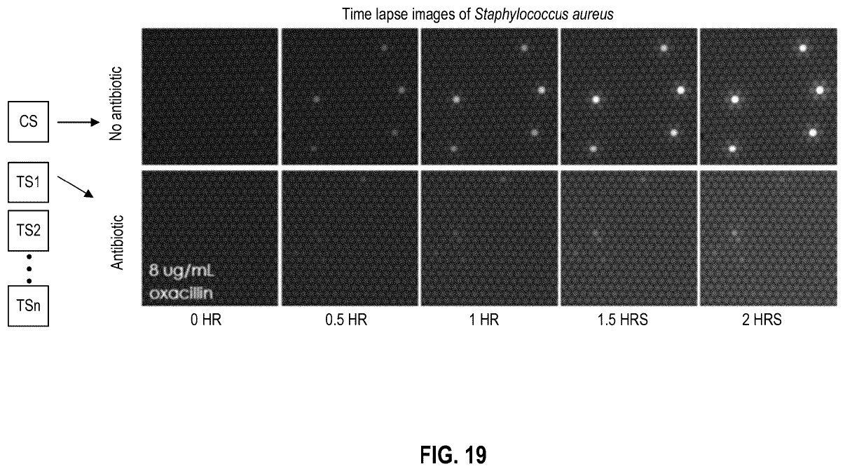

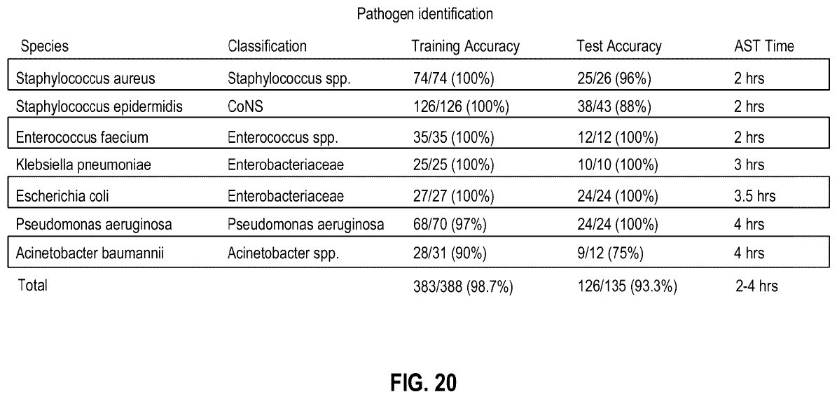

Patient Prognoses. In addition to patient diagnoses, the methods as generally described in FIG. 9 can be modified to provide patient prognostic information, such as patient morbidity, mortality, and hospital length of stay. FIG. 11 provides a schematic of the waveform signal chain. Both the control and test partition waveforms are fed into the same N1, CH, and CL but the resulting statistics are fed into separate input nodes of a predictor neural network (N2), allowing for a detailed comparison of the variation present in CS and TS. FIG. 12 illustrates one example of using this general scheme to provide a prognosis by testing or evaluating multiple drugs. In other aspects the methods can include various other sources of information, such as imaging data, biomarker, patient information, community information, and the like (e.g., see FIG. 13).

One advantage of the methods described herein is that a diagnosis can be derived using machine learning using sample partition statistics combined with other laboratory results, patient information, and community information to arrive at the most accurate and comprehensive diagnosis and prognosis in addition to those described in FIG. 10, including but not limited to:

Recommended Antimicrobial Drug. An antibiogram can provide clinicians with a set of antibiotics that will likely be effective against the microorganism causing the infection. It is still up to the clinician, however, to select the preferred antibiotic. An advantage of the methods described herein is that a diagnostic test can be modified to improve patient outcomes based on the phenotypic cell profile of the infection combined with relevant patient information, community information, imaging data, and disease markers. In a certain embodiment the diagnostic test can be designed to determine the final clinical decision that needs to be taken: which drug to administer and at which dose.

Patient information includes any information in the patient's health record and current condition, including but not limited to primary or secondary diagnoses, age, previous treatment history, and/or underlying conditions. Patient information can also include biometric data derived from a wearable or recorded in a personal application software.

Community information is the aggregate medical information related to a patient community, including, but not limited to genetic information; age; nationality; ethnicity; residence; place of work; socio-economic group; behavioral habit; lifestyle habit; environmental conditions; chemical exposure; pollutant and the like. If the patient has a microbial infection, community surveillance and hospital antibiogram information may play a big role in distinguishing between a set of antimicrobial candidates. In certain aspects the patient community can be limited to a group of patients at risk of, or currently or previously having a disease, condition, or combination thereof; a geographical region such as a neighborhood, city, county, state, country, continent, etc.; a demographic community having one or more common demographic; or the like.

Disease markers include any information that is relevant to the condition being tested that is derived from other laboratory tests including cytokines, microRNA, antibodies, cell-free DNA, human genomic information, blood chemistries etc., that can be used in combination with phenotypic cell data to improve diagnosis.

Imaging data includes patient sonogram, X-rays, CAT scan, PET scan, MRI, and the like. This type of data is typically interpreted by a clinician. In certain aspects raw imaging data can be encoded using a neural network to create a sparse representation and fed into the predictor neural net, e.g., N1, to aid in diagnosis.

The cell profile obtained from the methods described herein combined with patient history of antibiotic treatment could be used to predict the risk of a patient developing an antimicrobial resistant infection in the future, i.e., identifying a resistance risk. It can also be used in combination with community information to predict outbreak risk.

### I. Machine Learning and Deep Learning

Data scientists leverage machine learning techniques to build models that make predictions from real data. Typically, there are several pre-processing steps applied to raw data before machine learning models are applied to the data. Some examples of pre-processing steps include data quality processes (e.g., imputations and outlier removal), and feature extraction processes. Traditionally, such processes and models are built to work with either big data samples (“big data”) (e.g., a large number of data samples, for example terabytes or more of data that are too large to fit on a single machine and thus must be stored across multiple machines) or small data samples (“small data”) (e.g., a small number of data samples, for example kilobytes or megabytes of data that can be easily stored and processed on a single machine). Training sets are provided to a machine learning unit, such as a neural network or a support vector machine. Using the training set, the machine learning unit may generate a model to classify the sample according to components based on waveforms.

Artificial neural networks (NNets) mimic networks of “neurons” based on the neural structure of the brain. They process records one at a time, or in a batch mode, and “learn” by comparing their classification of the record (which, at the outset, is largely arbitrary) with the known actual classification of the record. In multilayer perceptron neural nets (MLP-NNets), the errors from the initial classification of the first record are fed back into the network and are used to modify the network's algorithm the second time around, and so on for many iterations.

In certain embodiments a fluorogenic response, or other signal, is measured over time and all measurement are collected into a set of waveforms. In certain aspects the waveforms can be clustered into known classes based on characteristic features using a neural network previously trained to recognize components in a sample. Certain aspects take a population of individual responses and use machine learning to extract clinically relevant information useful for diagnosis, treatment, and community health assessment and monitoring.

For example, providers have treated symptomatic patients with antibiotics while waiting for the culture results. This can lead to ineffective treatment, mistreatment, or over treatment of antibiotic which can have adverse consequences on both the individual patient and the population at large. Because aspects of this disclosure can be used to evaluate antimicrobial susceptibility that can be completed in 2 to 6 hours, effective treatment can begin much earlier.

Because this disclosure describes a machine learning technique to extract clinically relevant information from patient samples, the best models can be determined and refined without the bias of the architect. Hidden structure in the data can be uncovered by machine learning that may not be readily apparent to human observation.

For example, methods can be used to acquire pathogenic microbial samples at various levels of antibiotic resistance of all target pathogens and target antibiotics the assay is designed to test. This can be done by segmenting these samples into control samples and antibiotic susceptibility samples at various known levels of resistance. Let A be a random variable with Poisson distribution. For each control sample, compartmentalize the sample into discrete units that contain A microbial pathogen mixed with a fluorogenic agent, or other reagent. For each antibiotic susceptibility sample, and for each antibiotic compartmentalize the sample into discrete units that contain A microbial pathogens mixed with the antibiotic and a fluorogenic agent, or other reagent. Measure the fluorogenic response, or other signal, of each compartmentalized microbe over time and collect all signals into a set of waveforms. Partition the signals into two classes positive and negative waveforms based on an optical metric that effectively determines which compartments are populated with non-zero pathogens (positives) and which are empty (negatives).

Particular embodiments compartmentalize bacterial cells and resazurin dye inside oil droplets suspended in an aqueous emulsion. In this embodiment λ<1 is determined based on the dynamic range requirements of the assay. Droplets are loaded into an imaging chamber and a sequence of images are taken over a period of 2-6 hours. Droplets are identified in each image and are correlated over the sequence of images by software to produce waveforms of the mitochondrial resazurin/resorufin reduction. The software then segments the waveforms into “positives” and “negatives” using OTSU segmentation, kernel density estimation, or fixed threshold segmentation after applying an optical metric on each waveform. Additionally, in some embodiments the method filters any merged droplets, stacked droplets, and uncorrelated droplets.

After gathering waveform data, the data is partitioned into two sets for cross validation: a training set and a test set. Each positive waveform is normalized so that it has N points in the time dimension and its fluorescence dimension ranges from 0.0 to 1.0.

Training begins with an unsupervised learning step to reduce the dimension of each waveform from N points to a smaller number M=4 to 10. By normalizing in the fluorescence (or signal) dimension the relative magnitude of the curves are ignored and the shape of the curves become important. An auto-encoder is trained to reduce the dimensionality from N to a small number M=4 to 10. The training data is further segmented into a subset for training the auto-encoder and a subset for testing the auto-encoder. In some embodiments the auto-encoder is a neural network with a 1-D convolutional layer followed by a fully connected layer of size M. An activation function such as tanh is applied that puts the range of the output on 0.0 to 1.0. The output of these M activations becomes the “encoding” for each waveform in M dimensions. Other methods to reduce the dimensionality of the data exist such as principal component analysis and eigenvector decomposition exist and may be useful as well.

Next the optimal number of waveform “classes” K is determined. In some embodiments, the optimal value K is determined by iteratively applying “K means clustering” with various values of K. For each K, the distortion of the fit is computed, and the best value is chosen based on statistical methods such as the “elbow method” or rate distortion methods such as the “jump method” or “broken line method.” After the optimal value K is determined, the curves are classified according to nearest neighbor classification among the K cluster centers. This completes the unsupervised training step.

Training continues with a supervised step that models a regression function F(t,d)=μ mapping a target antibiotic and a digest of waveforms to an antibiotic susceptibility metric. In some embodiments the antibiotic susceptibility metric is the antibiotic minimum inhibitory concentration, and a digest of waveforms is a vector of 4(K+1) elements as follows:
(1) A vector of K+1 elements where each element 0<=j<k is the ratio of waveform class “j” occurring among the positive droplets in the control sample, and where the element at j=k is the ratio of the negatives occurring in the control sample.
(2) A vector of K+1 elements where elements are similarly defined as (1) except are the ratio of the average intensity of each class of droplet waveforms vs. the average intensity of all droplet waveforms in the control sample.
(3) A vector of K+1 elements where elements are similarly defined as (1) except from the antibiotic susceptibility test sample.
(4) A vector of K+1 elements where elements are similarly defined as (2) except from the antibiotic susceptibility test sample.

In some embodiments the model is a deep neural network with 4(K+1) input nodes, a number of hidden layers, and one output node corresponding to the antibiotic susceptibility metric. In some embodiments the antibiotic susceptibility metric is a unit-less value ranging from 0 to 1 where 0 denotes no antibiotic resistance and 1 denotes complete antibiotic resistance defined as no difference from the control. After the regression function is trained, the function is evaluated for accuracy by using the data withheld for the test data set.

In some embodiments the training data is augmented by:
(1) Constructing waveform digests on a random sampling of waveforms in each datum.
(2) Using M-nearest neighbor classification when classifying each positive waveform before constructing the waveform digests. For each positive waveform w, find the set M closest waveform cluster centers to w among all K. The vector v of K+1 elements j=0 to K+1 is a weighted average such that the element at position j in M is the amount of proximity w is to each center j, and that at position j not in M is 0. Examples of an “amount of proximity” could be an inverse distance weighting or evaluated from a probability distribution.
(3) A perturbation of the waveform digests by a random variable with small variance followed by a re-normalization.

When performing an antibiotic susceptibility test on a patient sample, the sample is loaded into A+1 circuits where circuit number A contains no antibiotic and is the control data, and each circuit number j=0 to A−1 contains a specific antibiotic j and is the antibiotic j's susceptibility data. The microbes are compartmentalized, the fluorogenic response (or other signal) is measured over time, and waveforms are extracted and segmented into “positives” and “negatives” as previously described. For each circuit j normalize and classify the waveforms. For each circuit j=0 to A−1, construct a digest d(j) as previously described containing the waveforms from circuits j and A. Evaluate the regression function F(j,d(j)) to determine the antibiotic susceptibility metric. Map this antibiotic susceptibility metric to a clinical result such as an SIR value or a MIC value based on a standard curve or table defined by the training data.

In certain aspects the Characterizer can receive a variety of inputs and send a variety of outputs depending on the goal of the analysis being performed. In certain examples of inputs and outputs into the Characterizer: outputs-(i) Average max signal of all the waveforms in a partition, (ii) Area under the curve for all the waveforms in a partition; (iii) Average max derivative for all the waveforms in a partition; (iv) Average point in time when each waveform crosses a particular threshold value for all the waveforms in a partition; (v) Same as above except using “median” instead of “average”; (vi) Same as above except for using normalized waveforms, where each waveform is divided by the average signal at the same time-point of the waveforms in a negative compartment (compartments without cells) within the same partition. Examples of inputs into the Classifier include (i) Raw waveforms and/or (ii) Dimensionally reduced waveforms, in addition to the above inputs with patient information, community information, and/or imaging data.

Furthermore, by using the auto-encoder to perform some unsupervised learning on the positive waveforms (the waveforms that actually show growth), the method can be used to derive clusters of data in the encoded vector space. The number of clusters might be more than the number of bacterial species if the distribution of some species is multi-modal in the encoded vector space. Call the number of clusters K and label them 1 through K inclusive. For each negative waveform (waveform that did not show growth) in the control partition, count it as belonging to cluster 0. For each positive waveform in the control partition, determine the cluster that it most likely belongs to.

Additional inputs into the characterizer include (i) A vector of K+1 elements j=0 to K where element j corresponds to the average of the max fluorescence values of all waveforms belonging to cluster j; (ii) A vector of K+1 elements j=0 to K where element j corresponds to the average area under the waveform fluorescence curve of all waveforms belonging to cluster j; (iii) A vector of K+1 elements j=0 to K where element j corresponds to the average of the max derivative fluorescence values of all waveforms belonging to cluster j; (iv) A vector of K+1 elements j=0 to K where element j corresponds to the average point in time each waveform fluorescence curve first attains a particular threshold value among all waveforms belonging to cluster j; (v) Similarly defined for “median” instead of “average”; (vi) Same as above except for using normalized waveforms, where each waveform is divided by the average signal at the same time-point of the waveforms in cluster 0 (compartments without cells) as well as others.

### II. General Methods

In certain aspects, the processing of a sample does not require lysis or washing. Since intact cells are used, only whole cells need to be manipulated rather than nucleic acid molecules, which are much more difficult manipulate due to their small size and propensity for charge-based interactions with different materials. Furthermore, the cells can be incubated at a single temperature, typically a relatively low temperature in the range of 25 to 45° C., obviating the need for thermal cycling equipment required for most NATs, which reduces cost and workflow complexity. Advantageously, by avoiding high temperature steps, the disclosure avoids significant issues that can arise with fluid evaporation and/or bubbles that can disrupt the integrity of the reaction and/or the fluorescent readout.

A test sample or a portion of a sample comprising at least one target cell can be compartmentalized, with or without being combined with a reagent, e.g., a viability dye or reporter, into compartments or droplets such that a statistically significant number of compartments or droplets contain no more than one cell or aggregation of cells (some cells tend to aggregate into cell clusters or chains). A reagent can be acted upon to produce or not produce a signal in the presence of a cell. Each droplet is monitored over time and the data used to identify and characterize each compartment. Further details on these processes are provided below.

Sample. Cells in the sample can include bacteria, fungi, plant cells, animal cells, or cells from any other cellular organism. The cells may be cultured cells or cells obtained directly from naturally occurring sources. The cells may be obtained directly from an organism or from a biological sample obtained from an organism, e.g., from sputum, saliva, urine, blood, cerebrospinal fluid, seminal fluid, stool, and tissue. In one embodiment the sample includes cells that are isolated from a biological sample comprising a variety of other components, such as other cells (background cells), viruses, proteins, and cell-free nucleic acids. The cells may be infected with a virus or another intracellular pathogen. The isolated cells may then be re-suspended in different media than those from which they were obtained. In one embodiment the sample comprises cells suspended in a nutrient medium that enables them to replicate and/or remain viable. The nutrient media may be defined media with known quantities or all ingredients or an undefined media where the nutrients are complex ingredients such yeast extract or casein hydrolysate, which contain a mixture of many chemical species of unknown proportions, including a carbon source such as glucose, water, various salts, amino acids and nitrogen. In one embodiment, the target cells in the test sample comprise pathogens and the nutrient media comprises a commonly used nutrient broth (liquid media) for culturing pathogens such as lysogeny broth, Mueller-Hinton broth, nutrient broth or tryptic soy broth. In any embodiment the media may be supplemented with a blood serum or synthetic serum to facilitate the growth of fastidious organisms.

Compartmentalization. Certain methods of the disclosure involve combining a sample or sample portion comprising a cell with one or more reagents and/or one or more reactants then compartmentalizing the sample or sample portion. The sample is compartmentalized such that a statistically significant portion of the compartments contain no more than one target cell or cell aggregate. The number of compartments can vary from hundreds to millions depending on the application. Compartment volumes can also vary between 1 μL to 10 pL depending on the application, for example between 25-500 pL. The methods described herein are compatible with any compartmentalization method.

One non-limiting method of compartmentalization is the use of droplets. While the methods for droplet formation differ, all the methods disperse an aqueous phase, the test sample in this case, into an immiscible phase, also referred to as the continuous phase, so that each droplet is surrounded by an immiscible carrier fluid. In one embodiment the immiscible phase is an oil. In certain aspects, the oil can comprise a surfactant. In a related embodiment, the immiscible phase is a fluorocarbon oil comprising a fluoro-surfactant. An advantage to using a fluorocarbon oil is that it is able to dissolve gases relatively well and it is biologically inert. Thus, the fluorocarbon oil used in the methods described herein comprises solubilized gases necessary for cell viability.

One non-limiting example of droplet formation is by using Laplace pressure gradients (see, for example, Dangla et al., 2013, PNAS 110(3):853-58). Laplace pressure is the differential pressure between the inside and outside of a curved surface, such as the difference in pressure between the inside and outside of a droplet. An aqueous phase containing cells or microbes can be introduced into a device having a reservoir of a continuous phase (i.e., immiscible fluid) forming an aqueous “tongue” in an appropriate device. The device can incorporate height variation(s) into a microchannel that subject the immiscible interfaces to a difference in curvature between the portion of the aqueous phase that has not encountered the height variation and the portion of the aqueous phase downstream of the height variation. As the aqueous phase flows through the height variation, a threshold curvature is reached for the portion of the aqueous phase downstream of the height variation beyond which the two portions cannot remain in static equilibrium, breaking off the aqueous phase into a droplet, as the downstream portion detaches from the tongue formed by introduction of the aqueous phase into a continuous phase, the size of the drops being determined by the device geometry. The height variation can be accomplished with a single step change in the height of a microchannel (step emulsification), multiple steps (multi-step emulsification), and a ramp or similarly gradual gradients of confinement.

Reporters. A variety of reporters may be used as reagents with the systems and methods disclosed herein. For example, a reporter can be a fluorophore, a protein labeled fluorophore, a protein comprising a photo oxidizable cofactor, a protein comprising another intercalated fluorophore, a mitochondrial vital stain or dye, a redox reactive dye, a membrane localizing dye, a dye with energy transfer properties, a pH indicating dye and/or any molecule that is composed of a dye and a moiety that can be acted upon by an enzyme. In a further aspect the reporter can be or include a resazurin dye, acridine, a tetrazolium dye, coumarin dye, an anthraquinone dye, a cyanine dye, an azo dye, a xanthene dye, an arylmethine dye, a pyrene derivative dye, a ruthenium bipyridyl complex dye, or derivatives thereof. Cell viability dyes, which are also included in the term reporter used herein, are used as analysis reagents to identify and characterize individual cells or pathogens encapsulated within droplets. Viability dyes have been used since the 1950's for cell viability purposes. However, these reagents are typically employed in samples that are significantly greater than 1 microliter in volume and/or are used in endpoint assays to indicate the presence of viable cells. Aspects of the disclosure use a viability dye in droplets that are between 1 μL and 100 nL, and more specifically 25-500 μL. In the method described herein the optical signal generated by the viability dye is concentrated by the small droplet volume and measured and recorded over an incubation time. In droplets containing viable cells, this results in an optical signature that is rapidly generated and has information about the characteristics of the cell encapsulated within the droplets. Combined with an environment stressor, such as an antimicrobial or cytotoxic drug, an additional signature can be generated by monitoring the optical signal of the droplets containing a cell over time. The optical signatures from the cell with and without the environmental stressor can be used to determine the identity and/or characteristics of the cell. Furthermore, the differences between the optical signatures obtained from a species of cells exposed to a drug compared to the optical signatures for same species of target cells that are not exposed to the drug can be used to determine the phenotypic drug resistance profile for the target cells obtained from a test sample. Because these signatures are generated from individual cells encapsulated in droplets, they represent information about the individual characteristics of each cell as opposed to an average characteristic of a population of cells that is generated from a bulk sample containing many cells.

The methods described herein are compatible with any viability dye or reporter or fluorogenic or luminogenic enzyme substrate that can be used with live cells (does not require cell lysis). In some embodiments the viability dye is a resorufin-based dye or derivative thereof. An example is resazurin which, when it is irreversibly reduced to pink and highly fluorescent resorufin (FIG. 6) it produces a fluorescent signal and a colorimetric shift (from blue to pink). In a preferred embodiment, the fluorescence is used because it offers better sensitivity over colorimetric signal changes. The limited-diffusion confinement within a sub-nanoliter volume of secreted fluorescent molecules quickly concentrates to detectable signal levels and is then detected by the methods described below. Furthermore, resorufin is reversibly reduced to non-fluorescent hydroresorufin (FIG. 6) if the redox environment dips below a particular redox threshold, usually around −100 mV. The combination of irreversible reduction from resazurin to resorufin and the reversible reduction of resorufin to hydroresorufin and oxidation of hydroresorufin back to resorufin depending on the redox potential of the droplet are what create the unique fluorescence signature over time in droplets that are small enough volume such that redox changes occur quickly in the presence of a single cell or cell aggregate. Examples of commercially available resazurin-based dyes are: AlamarBlue™ (various), PrestoBlue™ (Thermo Fisher Scientific), Cell-titer Blue™ (Promega), or Resazurin sodium salt powder (Sigma-Aldrich). Dyes that are structurally related to resazurin and can also be used in the method are: 10-acetyl-3, 7-dihydroxyphenoxazine (also known as Amplex Red™), C12 resazurin, and 1,3-dichloro-7-hydroxy-9,9-dimethylacridine-2(9H)-one (DDAO dye). In some embodiments, resorufin is modified with a cleavable moiety that acts as a fluorogenic substrate. Examples of commercially available resorufin-based fluorogenic substrates are 7-ethoxyresorufin, resorufin-β-D-glucoronic acid and resorufin-R-glucoronic-acid methyl ester. In some embodiments dyes that rely on tetrazolium-reduction, such as formazan dyes, can be used as the cell viability indicator. Examples include INT, MTT, XTT, MTS, TTC or tetrazolium chloride, NBT, and the WST series. In some embodiments, the reporter can include fluorogenic substrates such as 4-methylumbelliferone (4-MU), ethyl 7-hydroxycoumarin-3-carboxylate (EHC), 7-amido-4-methylcoumarin (AMC), fluorescein, and resorufin. In some embodiments, the reporter can be an Aldol® indicator (Biosynth). In some embodiments, the reporter can include luminogenic substrates such as luminol, Shaap deoxetanes, luciferin derivatives and proto-substrates, and dioxetane derivatives. Chemiluminescent reporters can be combined with fluorescent reporters to increase cell-specific waveform variation that aids in distinguishing similar DCCs. In certain aspects the reporter is a dioxetane derivative that carries a specific enzyme labile group. When exposed to an enzyme expressed by the isolated cell, the enzyme labile group is cleaved, liberating the corresponding unstable phenolate anion which decomposes, generating a high energy intermediate which emits light by returning to its unexcited ground state. An example of a dioxetane derivative is AquaSpark (Biosynth). Luciferase proto-substrates can also be combined with fluorescent and/or chemiluminescent reporters to increase cell-specific variation. The proto-substrate is reduced into a luciferase substrate within the isolated cell and then subsequently oxidized by a luciferase to produce a bioluminescent signal. Examples include Real-time Glo (Promega).

The disclosure provides for multiplexing of non-luminogenic, e.g., fluorescent, colorimetric, and/or luminogenic assays. As used herein, a “luminogenic assay” includes a reaction in which a molecule once acted on by a cellular component is luminogenic. Luminogenic assays include chemiluminescent and bioluminescent assays including but not limited to those which employ or detect luciferase, β-galactosidase, β-glucuronidase, β-lactamase, a protease, alkaline phosphatase, or peroxidase, and suitable corresponding substrates, e.g., modified forms of luciferin, coelenterazine, luminol, peptides or polypeptides, dioxetanes, dioxetanones, and related acridinium esters. As used herein, a “luminogenic assay reagent” includes a substrate, as well as an activator or enzyme that cleaves or modifies the substrate for a luminogenic reaction.

In certain aspects one or more isolated cells can harbor or express an enzyme useful in producing a luminogenic reaction. In particular, enzymes that are useful for such purposes include any protein that exhibits enzymatic activity, e.g., lipases, phospholipases, sulphatases, ureases, arylamidases, peptidases, proteases, oxidases, catalases, nitrate reductases, and esterases, including acid phosphatases, glucosidases, glucuronidases, galactosidases, carboxylesterases, and luciferases. In some embodiments, one of the enzymes is a hydrolytic enzyme. In some embodiments, at least two of the enzymes are hydrolytic enzymes. Examples of hydrolytic enzymes include alkaline and acid phosphatases, esterases, decarboxylases, phospholipase D, P-xylosidase, β-D-fucosidase, thioglucosidase, β-D-galactosidase, α-D-galactosidase, α-D-glucosidase, β-D-glucosidase, β-D-glucuronidase, α-D-mannosidase, β-D-mannosidase, β-D-fructofuranosidase, and β-D-glucosiduronase.

For alkaline phosphatase, in some embodiments the substrate includes a phosphate-containing dioxetane, such as 3-(2′-spiroadamantane)-4-methoxy-4-(3″-phosphoryloxy)phenyl-1,2-dioxetane, disodium salt, or disodium 3-(4-methoxyspiro[1,2-dioxetane-3,2′(5′-chloro)-tricyclo-[3.3.1.13,7]decan]-4-yl]phenyl phosphate, or disodium 2-chloro-5-(4-methoxyspiro{1,2-dioxetane-3,2′-(5′-chloro)-tricyclo {3.3.1.13,7]decan}-4-yl)-1-phenyl phosphate or disodium 2-chloro-5-(4-methoxyspiro{1,2-dioxetane-3,2′-tricyclo[3.3.1.13,7]decan}-4-yl)-1-phenzyl phosphate (AMPPD, CSPD, CDP-Star® and ADP-Star™, respectively).

For 3-galactosidase, in some embodiments the substrate includes a dioxetane containing galactosidase-cleavable or galactopyranoside group. The luminescence in the assay results from the enzymatic cleavage of the sugar moiety from the dioxetane substrate. Examples of such substrates include 3-(2′-spiroadamantane)-4-methoxy-4-(3″-β-D-galactopyranosyl)phenyl-1,2-dioxetane (AMPGD), 3-(4-methoxyspiro[1,2-dioxetane-3,2′-(5′-chloro)tricyclo[3.3.1.13,7]-decan]-4-yl-phenyl-β-D-galactopyranoside (Galacton®), 5-chloro-3-(methoxyspiro[1,2-dioxetane-3,2′-(5′-chloro)tricyclo[3.3.1]decan-4-yl-phenyl-β-D-galactopyranoside (Galacton-Plus®), and 2-chloro-5-(4-methoxyspiro[1,2-dioxetane-3,2′(5′-chloro)-tricyclo-[3.3.1.13,7]decan]-4-yl)phenyl β-D-galactopyranoside (Galacton-Star®).

In assays for 3-glucuronidase and 3-glucosidase, the substrate can include a dioxetane containing 3-glucuronidase-cleavable group such as a glucuronide, e.g., sodium 3-(4-methoxyspiro {1,2-dioxetane-3,2′-(5′-chloro)-tricyclo[3.3.1.13,7]decan}-4-yl)phenyl-β-D-glucuronate (Glucuron™). In assays for a carboxyl esterase, the substrate can include a suitable ester group bound to the dioxetane. In assays for proteases and phospholipases, the substrate can include a suitable enzyme-cleavable group bound to the dioxetane.

In some embodiments, the substrates for each enzyme in the assay are different. For assays which include one dioxetane containing substrate, the substrate optionally contains a substituted or unsubstituted adamantyl group, a Y group which may be substituted or unsubstituted and an enzyme cleavable group. Examples of dioxetanes include those mentioned above, e.g., those referred to as Galacton®, Galacton-Plus®, CDP-Star®, Glucuron™, AMPPD, Galacton-Star®, and ADP-Star™, as well as 3-(4-methoxyspiro{1,2-dioxetane-3,2′-(5′-chloro)-tricyclo[3.3.1.137]decan}-4-yl)phenyl-β-D-glucopyranoside (Glucon™), CSPD, disodium 3-chloro-5-(4-methoxyspiro{1,2-dioxetane-3,2′(5′-chloro)-tricyclo-[3.3.1.1]decan)-4-yl)-1-phenyl phosphate (CDP).

Cell (DCC) Aggregates. In some embodiments, the methods described are used for the diagnosis of microbial infections by identifying the microbes causing the infection and whether or not they are resistant to antimicrobial drugs. Thus, in this application, the DCCs can be single-celled microbes. Some bacteria, however, aggregate naturally into clusters or chains. In these cases, some droplets may comprise an aggregate of cells of the same microbial species (homogenous aggregate) rather than a single microbe. In these cases, the shape of the curve may be affected by the number of cells in the aggregate. However, the stored signature waveforms and call logic that are used to classify the compartmentalized cells can account for such aggregates the same way they can account for single cells. Furthermore, if the embodiment includes antimicrobial susceptibility testing the mixture comprising the antimicrobial drug will exhibit the same cell aggregation characteristics as the mixture that excludes the antimicrobial drug and the comparison will still be accurate. Therefore, while the method described herein generally comprises isolation of single cells in each droplet, it necessarily accommodates the case of a single cell species in a homogenous aggregate isolated in the droplet rather individual cells. In the case of cancer disease diagnosis, the target DCCs typically do not aggregate if they are circulating tumor cells. If the cancer cells are obtained from tissue, the tissue is typically disintegrated into individual cells prior to analysis. Therefore, each droplet will contain at most one cell; however, in some instances a cancer aggregate may also be analyzed using the described methods.

Signal Detection. Once the droplets have been generated, they are presented for analysis by an optical system, sensor, or sensor array. In some embodiments, the droplets are presented in a two-dimensional array so that thermal control can be maintained, and the droplet signals can be measured simultaneously (at a single instance in time) for many droplets. In the droplets containing target cells, the reporter will produce a concentrated fluorescent signal that will rise above the background droplets that do not contain cells. The concentrated signal of the droplet enables single cell identification in comparable time to standard PCR techniques which are the gold standard for fast identification. In certain aspects the signal is detected by exciting a reduced reporter with a specific wavelength of light and collecting the bandpass-filtered, Stokes-shifted light with a camera. The advantage to use imaging techniques is that they can image a droplet array that remains stationary and can therefore easily be monitored over time. Cytometry based methods typically employ endpoint detection instead of real time detection because of the difficulty in keeping track of the moving droplets over time. Another advantage to imaging the array is that all the droplets experience the same reaction conditions at the time of analysis. Therefore, droplet signals can be compared at equivalent time points which is advantageous since signals vary over time. With a cytometry approach, droplets pass by the detector at different times. Therefore, some droplets are incubated longer than others at the time of analysis. Finally, there may be different target cell species in the test sample. For each species, there may be an optimal droplet volume and dye or reporter concentration that maximizes signal at a particular time point. If an endpoint method is used, droplet volume and reporter concentrations do not need be controlled to the same degree because time can compensate for sub optimality and different species can be characterized universally within a single dye and droplet concentration.

Multiplexing. The methods described herein include the specific identification of multiple cells from a single test sample. By compartmentalizing single cells into their own isolated droplet, competition for resources between cells is eliminated. Therefore, individual cells that would exist collectively as a minority in a bulk population, now have equal access to nutrients when compared to the majority population of cells which results in a higher sensitivity for low abundance cells in a sample with multiple cell types. The multiplexing limitations for this invention depend on the ability to differentiate viability signatures between different cell types. Most methods for multiplexing require multiple dyes (fluorophores) which, in turn, require multiple sets of LEDs, excitation, and emission filters. Because the method described herein uses shape information rather than spectral information, the method can be used to multiplex many targets with a single dye or reporter requiring only one LED, emission filter, and excitation filter, thus simplifying the hardware needed to perform the analysis. In some embodiments, multiple reporters could be used in combination, together or separately, wherein reporters are selected from a set of different spectral wavelengths or luminogenic classes (e.g., fluorescence, chemiluminescence or colorimetric) such that multiple orthogonal metabolic pathways could be measured for each droplet of interest. For example, a redox sensitive fluorogenic dye such as resazurin could be used in combination with a second fluorogenic reporter for enzyme activity, such as fluorescein beta-galactopyranoside and a bioluminescent reporter such as luciferin/luciferase. Any number of additional reporters could be used in combination where each reporter would provide unique cell specific information.

### III. Reduction of Fluorescence Leakage

To prevent inter-droplet transfer of fluorescent molecules produced in a live cell reaction the disclosure utilizes a soluble complex with cyclodextrin contained within aqueous droplets. Various types of cyclodextrin can be used (e.g., α-cyclodextrin molecules, β-cyclodextrin molecules, or γ-cyclodextrin molecules). The formation of such a complex results in: (i) improved viability assay characteristics from the addition of cyclodextrin in bulk; and (ii) increased solubility of hydrophobic fluorescent molecules in the presence of cyclodextrin. Incorporation of such soluble complexes into live cell droplet assays enables the assay of live cells while retaining the fluorescent products within the droplets where they are produced.

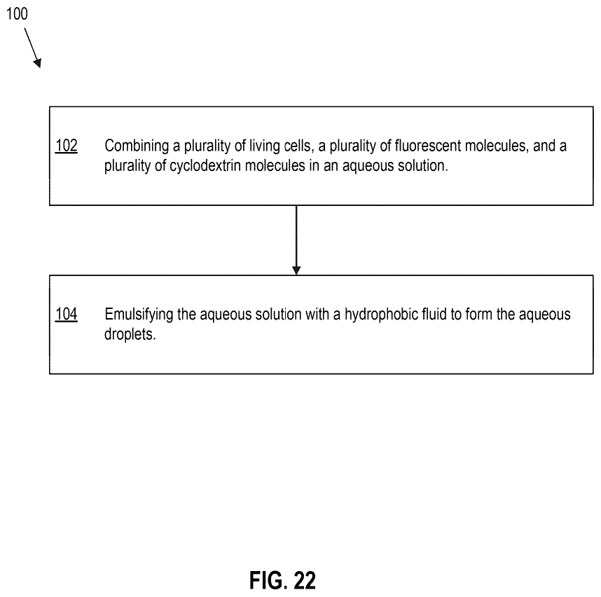

FIG. 22 is a flowchart illustrating an exemplary embodiment of method 100 for retaining fluorescent molecules within aqueous droplets suspended in an emulsion, consistent with the disclosed embodiments. The method begins with step 102.

Step 102 involves combining a plurality of living cells, a plurality of fluorescent molecules, and a plurality of cyclodextrin molecules in an aqueous solution. The living cells may be any type of cells, including bacterial cells, fungal cells, plant cells, and animal cells.

Step 104 involves emulsifying the aqueous solution with a hydrophobic fluid to form the aqueous droplets. The aqueous solution may be suspended in any suitable hydrophobic solution (e.g., oil) to create the emulsion. The droplets formed by the emulsion will include at least one of the living cells, (ii) at least one of the fluorescent molecules, and (iii) at least one cyclodextrin molecule. In some embodiments, to maintain the viability of the living cells, the temperature of the emulsion is kept below about 40° C.

### EXAMPLES

The following examples as well as the figures are included to demonstrate certain embodiments. It should be appreciated by those of skill in the art that the techniques disclosed in the examples or figures represent techniques discovered by the inventors to function well in the practice of the invention, and thus can be considered to constitute suitable modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Core Technology

Individual cells are encapsulated into pico-scale droplets with a cell viability dye that becomes fluorescent, or in some embodiments loses fluorescence, in the presence of a live cell. In certain embodiments a resazurin-based dye is used. In the presence of a live cell, resorufin molecules will rapidly concentrate within the picoscale droplet environment and produce an easily detectable fluorescent signal.

Cyclodextrin is also encapsulated with the cell viability dye and the individual cells. Cyclodextrin does not affect the conversion of non-fluorescent to fluorescent molecules, or vice versa, in the presence of a live cell and mitigates the leakage of the fluorescent product outside of the droplet.

The rate at which each cell reduces resazurin depends on cell-specific characteristics such as permeability, metabolic profile, size, and growth rate. Furthermore, within the pico-scale environment inside the droplet, the encapsulated cell will dominate the conditions (e.g., redox potential) that determine whether resorufin will reduce to hydroresorufin. The result is that different cell types (e.g., different microbial species) produce unique fluorescent “signatures” over time allowing for the identification of the cell type in each droplet.

Additional fluorogenic reagents may also be incorporated into these assays as needed to increase signal variation and thereby add specificity to the signatures as needed. Moreover, for microbial cells, antimicrobial susceptibility (AST) can be measured by introducing an antimicrobial drug into the droplets and comparing the viability signals from those droplets that contain an antimicrobial drug to those that do not contain an antimicrobial drug.

The reactions are observed in real-time by arranging tens-of-thousands of droplets into a two-dimensional array and recording the fluorescence from each droplet using a wide-field imaging system with LED excitation and CMOS sensor detection.

Neural net powered by deep learning. Machine learning can be used to recognize cells (e.g., microbes) by their fluorescent signatures and to determine drug susceptibility. Specifically, a proprietary deep neural network architecture can be used to interpret identification (ID) and antibiotic susceptibility (AST) results. By leveraging machine learning, bacteria can be classified based on phenotypic differences apparent at single-cell resolution, allowing seamless integration of pathogen identification and antibiotic susceptibility using the same detection modality. This discovery uniquely allows reduced test costs and complexity. The neural net input data is based on time-series images from a two-dimensional droplet array. The proprietary software identifies and tracks the droplets over time and generates a per-droplet waveform of fluorescence intensity with respect to time.

For the purposes of bacterial ID, only “control” circuits with no antibiotic are used. Each droplet is classified and total droplets for each species are counted. For each antibiotic, a control circuit where no antibiotic is present is compared to the test circuit where an antibiotic is present at a specified concentration. The neural network will determine the effectiveness of the antibiotic by comparing how the waveforms change between the control and antibiotic test circuits.

Pathogen Identification. Taken together, ID and AST provide clinicians with the definitive information required to precisely treat a condition (e.g., a bacterial infection). The value of pathogen identification depends on when AST results become available. Under the current paradigm, ID results are available hours, sometimes days before AST results are available. Without timely AST results, there is an increased burden on ID to provide speciation in order to help clinicians adjust the antibiotic regimen.

For example, in the absence of an AST result, a clinician will want to distinguish between Acinetobacter baumannii and other Acinetobacter species because A. baumannii is often resistant to certain antibiotics that the other Acinetobacters are not.

However, if the AST results are available at the same time as the ID results there is no need to distinguish between A. baumannii and other Acinetobacters because the full resistance profile is revealed and, according to the CLSI guidelines, the actions guided by AST results are identical for all Acinetobacter species.

When ID and AST results are available simultaneously, the ID result is used to interpret the AST result according to CLSI guidelines, producing an antibiogram for the infection (an antibiogram is a list of the antibiotics that were tested and whether the infection is susceptible (S), intermediate (1), or resistant (R) to each listed antibiotic). This is widely considered the most clinically important test result in the microbiology laboratory.

### Example 2: Cyclodextrin Mitigated the Leakage of the Fluorescent Product Outside of the Droplet

Growth media, bacterial cells, viability indicator and cyclodextrin were combined in bulk (dispersed phase) and dispensed into a microfluidic circuit containing oil and surfactant (continuous phase). In a separate reaction, the same growth media, bacteria cells, and viability indicator (without the cyclodextrin) were dispensed into a separate microfluidic circuit containing the same continuous phase. Each dispersed phase was then compartmentalized within the respective microfluidic circuits to form several thousand picoliter scale droplets into two-dimensional droplet arrays. The droplets were incubated at a constant temperature of 38° C. for 6 hours. Every 2 minutes, an image was taken capturing the fluorescence intensity of each droplet. At the end of the 6 hour incubation, 144 images were used to generate droplet intensity waveforms as a function of time.

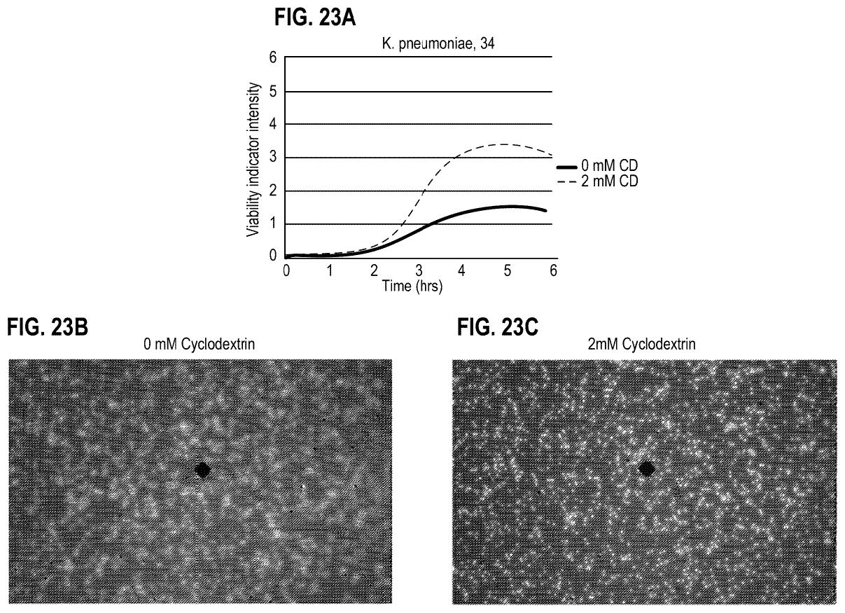

The results of these experiments are presented in FIGS. 23-26, which reveal that cyclodextrin prevented leakage of the fluorescent reporter outside of the droplet. Specifically, FIG. 23A-C demonstrated the decreased leakage of the fluorescent marker from the addition of 2 mM of cyclodextrin in droplets containing Klebsiella pneumoniae cells. FIG. 23A is a graphical representation of the increased viability indicator intensity for droplets containing 2 mM cyclodextrin compared to droplets lacking cyclodextrin. Additionally, the intensity of fluorescence of the individual droplets was significantly higher with the addition of 2 mM of cyclodextrin (FIG. 23B) than with no cyclodextrin added (FIG. 23C). Further, the increased contrast seen with the addition of 2 mM of cyclodextrin (FIG. 23B) compared to no cyclodextrin added (FIG. 23C) confirms that the fluorescent molecules remained within their respective droplets when cyclodextrin was added.

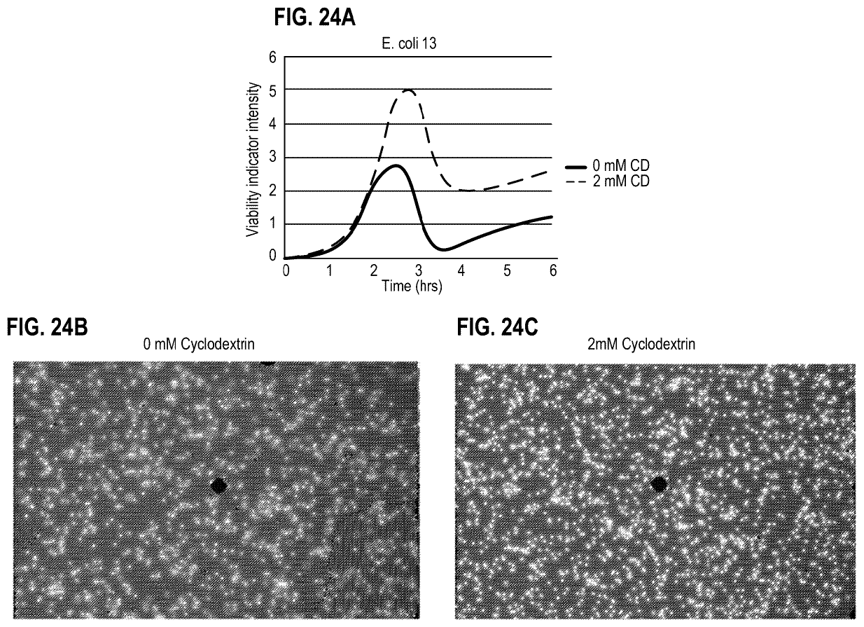

FIG. 24A-C demonstrated the decreased leakage of the fluorescent marker from the addition of 2 mM of cyclodextrin in droplets containing Escherichia coli cells. FIG. 24A is a graphical representation of the increased viability indicator intensity for droplets containing 2 mM cyclodextrin compared to droplets lacking cyclodextrin. Additionally, the intensity of fluorescence of the individual droplets was significantly higher with the addition of 2 mM of cyclodextrin (FIG. 24B) than with no cyclodextrin added (FIG. 24C). Further, the increased contrast seen with the addition of 2 mM of cyclodextrin (FIG. 24B) compared to no cyclodextrin added (FIG. 24C) confirms that the fluorescent molecules remained within their respective droplets when cyclodextrin was added.

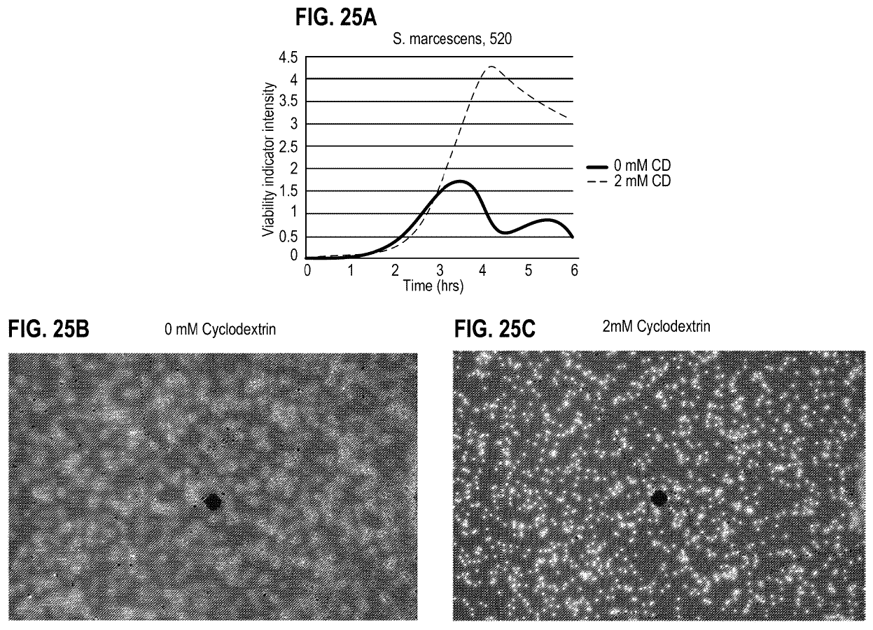

FIG. 25A-C demonstrated the decreased leakage of the fluorescent marker from the addition of 2 mM of cyclodextrin in droplets containing a first strain of Serratia marcescens cells. FIG. 25A is a graphical representation of the increased viability indicator intensity for droplets containing 2 mM cyclodextrin compared to droplets lacking cyclodextrin. Additionally, the intensity of fluorescence of the individual droplets was significantly higher with the addition of 2 mM of cyclodextrin (FIG. 25B) than with no cyclodextrin added (FIG. 25C). Further, the increased contrast seen with the addition of 2 mM of cyclodextrin (FIG. 25B) compared to no cyclodextrin added (FIG. 25C) confirms that the fluorescent molecules remained within their respective droplets when cyclodextrin was added.

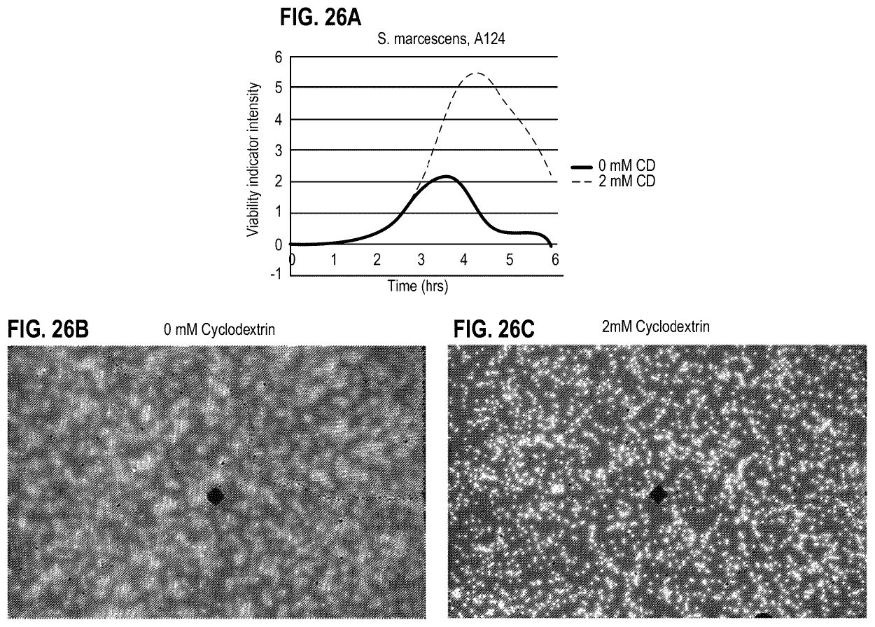

FIG. 26A-C demonstrated the decreased leakage of the fluorescent marker from the addition of 2 mM of cyclodextrin droplets containing of a second strain of Serratia marcescens cells. FIG. 26A is a graphical representation of the increased viability indicator intensity for droplets containing 2 mM cyclodextrin compared to droplets lacking cyclodextrin. Additionally, the intensity of fluorescence of the individual droplets was significantly higher with the addition of 2 mM of cyclodextrin (FIG. 26B) than with no cyclodextrin added (FIG. 26C). Further, the increased contrast seen with the addition of 2 mM of cyclodextrin (FIG. 26B) compared to no cyclodextrin added (FIG. 26C) confirms that the fluorescent molecules remained within their respective droplets when cyclodextrin was added.

The beneficial increase in fluorescence intensity within droplets and decrease in fluorescence leakage between droplets is seen when using other types of living cells. Examples of other types of cells include other bacterial cells, fungal cells, plant cells, and animal cells.

### Recitation of Embodiments

Embodiment 1: A method for retaining fluorescent molecules within aqueous droplets suspended in an emulsion, the method comprising: combining a plurality of living cells, a plurality of fluorescent molecules, and a plurality of cyclodextrin molecules in an aqueous solution; and emulsifying the aqueous solution with a hydrophobic fluid to form the aqueous droplets; wherein at least one of the aqueous droplets includes (i) a single living cell or a single cell species in a homogenous aggregate of the plurality of living cells, (ii) at least one fluorescent molecule of the plurality of fluorescent molecules, and (iii) at least one cyclodextrin molecule of the plurality of cyclodextrin molecules.

Embodiment 2: The method of embodiment 1, wherein the plurality of living cells includes at least one of bacterial cells, fungal cells, plant cells, and animal cells.

Embodiment 3: The method of any of embodiments 1 or 2, wherein the plurality of living cells includes living cells of at least one of Klebsiella pneumoniae, Escherichia coli, and Serratia marcescens.

Embodiment 4: The method of any of embodiments 1 or 2, wherein the plurality of living cells includes human cells.

Embodiment 5: The method of embodiment 4, wherein the human cells are derived from sputum, saliva, urine, blood, cerebrospinal fluid, seminal fluid, stool, or tissue.

Embodiment 6: The method of any of embodiments 1-5, wherein the hydrophobic fluid comprises an oil.

Embodiment 7: The method of embodiment 6, wherein the oil is a fluorocarbon oil.

Embodiment 8: The method of any of embodiments 1-7, wherein the fluorescent molecules are hydrophobic fluorescent molecules.

Embodiment 9: The method of embodiment 8, wherein the hydrophobic fluorescent molecules are resazurin dye molecules, acridine molecules, tetrazolium dye molecules, coumarin dye molecules, anthraquinone dye molecules, cyanine dye molecules, azo dye molecules, xanthene dye molecules, arylmethine dye molecules, pyrene derivative dye molecules, or ruthenium bipyridyl complex dye molecules.

Embodiment 10: The method of any of embodiments 1-9, wherein the cyclodextrin molecules are α-cyclodextrin molecules, β-cyclodextrin molecules, or γ-cyclodextrin molecules.

Embodiment 11: The method of any of embodiments 1-10, wherein the fluorescent molecules and the cyclodextrin molecules do not impede viability or metabolic activity of the living cells.

Embodiment 12: The method of any of embodiments 1-11, wherein the combining and the emulsifying are performed at a temperature of no greater than 40° C.

Embodiment 13: The method of any of embodiments 1-12, further comprising combining a surfactant in the aqueous solution.

Embodiment 14: The method of embodiment 13, wherein the surfactant is a fluoro-surfactant.

Embodiment 15: An emulsion comprising a plurality of aqueous droplets suspended in a hydrophobic fluid, wherein at least one of the aqueous droplets includes: a single living cell or a single cell species in a homogenous aggregate; at least one fluorescent molecule; and at least one cyclodextrin molecule.

Embodiment 16: The emulsion of embodiment 15, wherein the single living cell or the single cell species in a homogenous aggregate includes one of bacterial cells, fungal cells, plant cells, and animal cells.

Embodiment 17: The emulsion of any of embodiments 15 or 16, wherein the single living cell or the single cell species in a homogeneous aggregate includes one of Klebsiella pneumoniae, Escherichia coli, and Serratia marcescens.

Embodiment 18: The emulsion of any of embodiments 15 or 16, wherein the single living cell or the single cell species in a homogeneous aggregate includes human cells.

Embodiment 19: The emulsion of embodiment 18, wherein the human cells are derived from sputum, saliva, urine, blood, cerebrospinal fluid, seminal fluid, stool, or tissue.

Embodiment 20: The emulsion of any of embodiments 15-19, wherein the hydrophobic fluid comprises an oil.

Embodiment 21: The emulsion of embodiment 20, wherein the oil is a fluorocarbon oil.

Embodiment 22: The emulsion of any of embodiments 15-21, wherein the fluorescent molecules are hydrophobic fluorescent molecules.

Embodiment 23: The emulsion of embodiment 22, wherein the hydrophobic fluorescent molecules are resazurin dye molecules, acridine molecules, tetrazolium dye molecules, coumarin dye molecules, anthraquinone dye molecules, cyanine dye molecules, azo dye molecules, xanthene dye molecules, arylmethine dye molecules, pyrene derivative dye molecules, or ruthenium bipyridyl complex dye molecules.

Embodiment 24: The emulsion of any of embodiments 15-23, wherein the at least one cyclodextrin molecule is at least one α-cyclodextrin molecule, at least one β-cyclodextrin molecule, or at least one γ-cyclodextrin molecule.

Embodiment 25: The emulsion of any of embodiments 15-24, wherein the fluorescent molecules and the cyclodextrin molecules are (i) biocompatible with the single living cell or the single cell species in a homogenous aggregate, and (ii) do not impede metabolic activity of the single living cell or the single cell species in a homogenous aggregate.

Embodiment 26: The emulsion of any of embodiments 15-25, wherein the at least one of the aqueous droplets further includes a surfactant.

Embodiment 27: The emulsion of embodiment 26, wherein the surfactant is a fluoro-surfactant.

The foregoing description has been presented for purposes of illustration. It is not exhaustive and does not limit the invention to the precise forms or embodiments disclosed. Modifications and adaptations of the invention will be apparent to those skilled in the art from consideration of the specification and practice of the disclosed embodiments of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for retaining fluorescent molecules within aqueous droplets suspended in an emulsion, the method comprising:
combining a plurality of living cells, a plurality of fluorescent molecules, and a plurality of cyclodextrin molecules in an aqueous solution; and
emulsifying the aqueous solution with a hydrophobic fluid to form the aqueous droplets;
wherein at least one of the aqueous droplets includes (i) a single living cell or a single cell species in a homogenous aggregate of the plurality of living cells, (ii) at least one fluorescent molecule of the plurality of fluorescent molecules, and (iii) at least one cyclodextrin molecule of the plurality of cyclodextrin molecules.

2. The method of claim 1, wherein the plurality of living cells includes at least one of bacterial cells, fungal cells, plant cells, and animal cells.

3. The method of any of claim 1 or 2, wherein the plurality of living cells includes living cells of at least one of Klebsiella pneumoniae, Escherichia coli, and Serratia marcescens.

4. The method of any of claim 1 or 2, wherein the plurality of living cells includes human cells.

5. The method of claim 4, wherein the human cells are derived from sputum, saliva, urine, blood, cerebrospinal fluid, seminal fluid, stool, or tissue.

6. The method of any of claims 1-5, wherein the hydrophobic fluid comprises an oil.

7. The method of claim 6, wherein the oil is a fluorocarbon oil.

8. The method of any of claims 1-7, wherein the fluorescent molecules are hydrophobic fluorescent molecules.

9. The method of claim 8, wherein the hydrophobic fluorescent molecules are resazurin dye molecules, acridine molecules, tetrazolium dye molecules, coumarin dye molecules, anthraquinone dye molecules, cyanine dye molecules, azo dye molecules, xanthene dye molecules, arylmethine dye molecules, pyrene derivative dye molecules, or ruthenium bipyridyl complex dye molecules.

10. The method of any of claims 1-9, wherein the cyclodextrin molecules are α-cyclodextrin molecules, β-cyclodextrin molecules, or γ-cyclodextrin molecules.

11. The method of any of claims 1-10, wherein the fluorescent molecules and the cyclodextrin molecules do not impede viability or metabolic activity of the living cells.

12. The method of any of claims 1-11, wherein the combining and the emulsifying are performed at a temperature of no greater than 40° C.

13. The method of any of claims 1-12, further comprising combining a surfactant in the aqueous solution.

14. The method of claim 13, wherein the surfactant is a fluoro-surfactant.

15. An emulsion comprising a plurality of aqueous droplets suspended in a hydrophobic fluid, wherein at least one of the aqueous droplets includes:
a single living cell or a single cell species in a homogenous aggregate;
at least one fluorescent molecule; and
at least one cyclodextrin molecule.

16. The emulsion of claim 15, wherein the single living cell or the single cell species in a homogenous aggregate includes one of bacterial cells, fungal cells, plant cells, and animal cells.

17. The emulsion of any of claim 15 or 16, wherein the single living cell or the single cell species in a homogeneous aggregate includes one of Klebsiella pneumoniae, Escherichia coli, and Serratia marcescens.

18. The emulsion of any of claim 15 or 16, wherein the single living cell or the single cell species in a homogeneous aggregate includes human cells.

19. The emulsion of claim 18, wherein the human cells are derived from sputum, saliva, urine, blood, cerebrospinal fluid, seminal fluid, stool, or tissue.

20. The emulsion of any of claims 15-19, wherein the hydrophobic fluid comprises an oil.

21. The emulsion of claim 20, wherein the oil is a fluorocarbon oil.

22. The emulsion of any of claims 15-21, wherein the fluorescent molecules are hydrophobic fluorescent molecules.

23. The emulsion of claim 22, wherein the hydrophobic fluorescent molecules are resazurin dye molecules, acridine molecules, tetrazolium dye molecules, coumarin dye molecules, anthraquinone dye molecules, cyanine dye molecules, azo dye molecules, xanthene dye molecules, arylmethine dye molecules, pyrene derivative dye molecules, or ruthenium bipyridyl complex dye molecules.

24. The emulsion of any of claims 15-23, wherein the at least one cyclodextrin molecule is at least one α-cyclodextrin molecule, at least one β-cyclodextrin molecule, or at least one γ-cyclodextrin molecule.

25. The emulsion of any of claims 15-24, wherein the fluorescent molecules and the cyclodextrin molecules are (i) biocompatible with the single living cell or the single cell species in a homogenous aggregate, and (ii) do not impede metabolic activity of the single living cell or the single cell species in a homogenous aggregate.

26. The emulsion of any of claims 15-25, wherein the at least one of the aqueous droplets further includes a surfactant.

27. The emulsion of claim 26, wherein the surfactant is a fluoro-surfactant.
